SEQUENCE LISTING (40 sequences)

SEQ ID NO: 1 - protein, 858 aa, synthetic construct
GTENLTDITFFNTHGIKPSDHPMWEQQLELEAEMRSAGIARFRSMLEKSEQRGMMTGVASVRRLMVHAHAKTVEAFHAFMAEVKSSKAGRRHVAAAYLADADPDVACHLTLRALLDTVSQQMKLANAASRVATLIEDELHFNAFREQNEGAYNWSRRAILERSQNRNYQRRVMNKLARNRQIQWEDWTDEIKVRVGTKMIELVVEATGLFELRRISDGRNFTNIYVTATQETLDWIKTENSRLEPMSPIYLPCLMPPRPWTSPVSGGYWSGRVRNLRLVKTHNRAYLEDLENADMQTVYDAVNAMQNTAWQINRPVYEVMRTLWDNQSTLGNLPMSEDEPLPEAPLWLEPGMTKEDMTPDQLEQFMRWKAERAAIYETNAKAVSKRLQFSRMLWVAERFKDVEEFYFPYQLDFRGRVYAVPLFLNPQGNDAARGLLTFANTVPITDQDGADWLAIHGASLWGVDKVGLQERVEWVIENEAAILASAENPYDNRFWATAEKPWQALAFCFEWAGFKREGFAFHSSLPVQMDGTCNGLQNFSAMLLDEIGGRAVNLVPGEKPNDIYQTVADVVVEKLQQIAAACPEDTTEVETKDGGTRTVDSEGALARKWLAYGVTRKVTKRPVMTLAYGATKFGFRQQVFDDIVSEWRAEAKDAFPFEGAGWEAARFMGDLIWQCVGEVVVAARGAMDWLQEVAKIASKQRLPVIWTAPTGFKVMQEYTTAEQKRLELTFQKVRLRLQIDTSTKKLDVRKQSSGISPNWVHSMDAAHMQRTVARCHAEGIRSFSLIHDSYGTHAGNAWAMAQFLREEFVRMYSEHDVLAEFREEIAMLLGVEPEALPKLPPKGSLDLHQVLESDFFFA

SEQ ID NO: 2 - protein, 858 aa, synthetic construct
GTENLTDITFFNTHGIKPSDHPMWEQQLELEAEMRSAGIARFRSMLEKSEQRGMMTGVASVRRLMVHAHAKTVEAFHAFMAEVKSSKAGRRHVAAAYLADADPDVACHLTLRALLDTVSQQMKLANAASRVATLIEDELHFNAFREQNEGAYNWSRRAILERSQNRNYQRRVMNKLARNRQIQWEDWTDEIKVRVGTKMIELVVEATGLFELRRMSDGRNFTNIYVTATQETLDWIKTENSRLEPMSPIYLPCLMPPRPWTSPVSGGYWSGRVRNLRLVKTHNRAYLEDLENADMQTVYDAVNAMQNTAWQINRPVYEVMRTLWDNQSTLGNLPMSEDEPLPEAPLWLEPGMTKEDMTPDQLEQFMRWKAERAAIYETNAKAVSKRLQFSRMLWVAERFKDVEEFYFPYQLDFRGRVYAVPLFLNPQGNDAARGLLTFANTVPITDQDGADWLAIHGANLWGVDKVGLQERVEWVIENEAAILASAENPYDNRFWATAEKPWQALAFCFEWAGFKREGFAFHSSLPVQMDGTCNGLQNFSAMLLDEIGGRAVNLVPGEKPNDIYQTVADVVVEKLQQIAAACPEDTTEVETKDGGTRTVDSEGALARKWLAYGVTRKVTKRPVMTLAYGATKFGFRQQVFDDIVSEWRAEAKDAFPFEGAGWEAARFMGDLIWQCVGEVVVAARGAMDWLQEVAKIASKQRLPVIWTAPTGFKVMQEYTTAEQKRLELTFQKVRLRLQIDTSTKKLDVRKQSSGISPNWVHSMDAAHMQRTVARCHAEGIRSFSLIHDSYGTHAGNAWAMAQFLREEFVRMYSEHDVLAEFREEIAMLLGVEPEALPKLPPKGSLDLHQVLESDFFFA

SEQ ID NO: 3 - protein, 858 aa, synthetic construct
GTENLTDITFFNTHGIKPSDHPMWEQQLELEAEMRSAGIARFRSMLEKSEQRGMMTGVASVRRLMVHAHAKTVEAFHAFMAEVKSSKAGRRHVAAAYLADADPDVACHLTLRALLDTVSQQMKLANAASRVATLIEDELHFNAFREQNEGAYNWSRRAILERSQNRNYQRRVMNKLARNRQIQWEDWTDEIKVRVGTKMIELVVEATGLFELRRMSDGRNFTNIYVTATQETLDWIKTENSRLEPMSPIYLPCLMPPRPWTSPVSGGYWSGRVRNLRLVKTHNRAYLEDLENADMQTVYDAVNAMQNTAWQINRPVYEVMRTLWDNQSTLGNLPMSEDEPLPEAPLWLEPGMTKEDMTPDQLEQFMRWKAERAAIYETNAKAVSKRLQFSRMLWVAERFKDVEEFYFPYQLDFRGRVYAVPLFLNPQGNDAARGLLTFANTVPITDQDGADWLAIHGASLWGVDKVGLQERVEWVIENEAAILASAENPYDNRFWATAEKPWQALAFCFEWAGFKREGFAFHSSLPVQMDGTCNGLQHFSAMLLDEIGGRAVNLVPGEKPNDIYQTVADVVVEKLQQIAAACPEDTTEVETKDGGTRTVDSEGALARKWLAYGVTRKVTKRPVMTLAYGATKFGFRQQVFDDIVSEWRAEAKDAFPFEGAGWEAARFMGDLIWQCVGEVVVAARGAMDWLQEVAKIASKQRLPVIWTAPTGFKVMQEYTTAEQKRLELTFQKVRLRLQIDTSTKKLDVRKQSSGISPNWVHSMDAAHMQRTVARCHAEGIRSFSLIHDSYGTHAGNAWAMAQFLREEFVRMYSEHDVLAEFREEIAMLLGVEPEALPKLPPKGSLDLHQVLESDFFFA

SEQ ID NO: 4 - protein, 858 aa, synthetic construct
GTENLTDITFFNTHGIKPSDHPMWEQQLELEAEMRSAGIARFRSMLEKSEQRGMMTGVASVRRLMVHAHAKTVEAFHAFMAEVKSSKAGRRHVAAAYLADADPDVACHLTLRALLDTVSQQMKLANAASRVATLIEDELHFNAFREQNEGAYNWSRRAILERSQNRNYQRRVMNKLARNRQIQWEDWTDEIKVRVGTKMIELVVEATGLFELRRMSDGRNFTNIYVTATQETLDWIKTENSRLEPMSPIYLPCLMPPRPWTSPVSGGYWSGRVRNLRLVKTHNRAYLEDLENADMQTVYDAVNAMQNTAWQINRPVYEVMRTLWDNQSTLGNLPMSEDEPLPEAPLWLEPGMTKEDMTPDQLEQFMRWKAERAAIYETNAKAVSKRLQFSRMLWVAERFKDVEEFYFPYQLDFRGRVYAVPLFLNPQGNDAARGLLTFANTVPITDQDGADWLAIHGASLWGVDKVGLQERVEWVIENEAAILASAENPYDNRFWATAEKPWQALAFCFEWAGFKREGFAFHSSLPVQMDGTCNGLQNFSAMLLDEIGGRAVNLVPGEKPNDIYQTVADVVVEKLQQIAAACPEDTTEVETKDGGTRTVDSEGALARKWLAYGVTRKVTKRPVMTLAYGATKFGFRQQVFDDIVSEWRAEAKDAFPFEGAGWEAARFMGDLIWQCVGEVVVAARGAMDWLQEVAKIASKQRLPVIWTAPTGFKVMQEYTTAEQKRLELTFQKVRLRLQIDTSTKKLDVRKQSSGISPNFVHSMDAAHMQRTVARCHAEGIRSFSLIHDSYGTHAGNAWAMAQFLREEFVRMYSEHDVLAEFREEIAMLLGVEPEALPKLPPKGSLDLHQVLESDFFFA

SEQ ID NO: 5 - protein, 858 aa, synthetic construct
GTENLTDITFFNTHGIKPSDHPMWEQQLELEAEMRSAGIARFRSMLEKSEQRGMMTGVASVRRLMVHAHAKTVEAFHAFMAEVKSSKAGRRHVAAAYLADADPDVACHLTLRALLDTVSQQMKLANAASRVATLIEDELHFNAFREQNEGAYNWSRRAILERSQNRNYQRRVMNKLARNRQIQWEDWTDEIKVRVGTKMIELVVEATGLFELRRMSDGRNFTNIYVTATQETLDWIKTENSRLEPMSPIYLPCLMPPRPWTSPVSGGYWSGRVRNLRLVKTHNRAYLEDLENADMQTVYDAVNAMQNTAWQINRPVYEVMRTLWDNQSTLGNLPMSEDEPLPEAPLWLEPGMTKEDMTPDQLEQFMRWKAERAAIYETNAKAVSKRLQFSRMLWVAERFKDVEEFYFPYQLDFRGRVYAVPLFLNPQGNDAARGLLTFANTVPITDQDGADWLAIHGASLWGVDKVGLQERVEWVIENEAAILASAENPYDNRFWATAEKPWQALAFCFEWAGFKREGFAFHSSLPVQMDGTCNGLQNFSAMLLDEIGGRAVNLVPGEKPNDIYQTVADVVVEKLQQIAAACPEDTTEVETKDGGTRTVDSEGALARKWLAYGVTRKVTKRPVMTLAYGATKFGFRQQVFDDIVSEWRAEAKDAFPFEGAGWEAARFMGDLIWQCVGEVVVAARGAMDWLQEVAKIASKQRLPVIWTAPTGFKVMQEYTTAEQKRLELTFQKVRLRLQIDTSTKKLDVRKQSSGISPNWVHSMDAAHMQRTVARCHAEGIRSFSLIHDSYGTHAGNADAMAQFLREEFVRMYSEHDVLAEFREEIAMLLGVEPEALPKLPPKGSLDLHQVLESDFFFA

SEQ ID NO: 6 - protein, 858 aa, synthetic construct
GTENLTDITFFNTHGIKPSDHPMWEQQLELEAEMRSAGIARFRSMLEKSEQRGMMTGVASVRRLMVHAHAKTVEAFHAFMAEVKSSKAGRRHVAAAYLADADPDVACHLTLRALLDTVSQQMKLANAASRVATLIEDELHFNAFREQNEGAYNWSRRAILERSQNRNYQRRVMNKLARNRQIQWEDWTDEIKVRVGTKMIELVVEATGLFELRRMSDGRNFTNIYVTATQETLDWIKTENSRLEPMSPIYLPCLMPPRPWTSPVSGGYWSGRVRNLRLVKTHNRAYLEDLENADMQTVYDAVNAMQNTAWQINRPVYEVMRTLWDNQSTLGNLPMSEDEPLPEAPLWLEPGMTKEDMTPDQLEQFMRWKAERAAIYETNAKAVSKRLQFSRMLWVAERFKDVEEFYFPYQLDFRGRVYAVPLFLNPQGNDAARGLLTFANTVPITDQDGADWLAIHGASLWGVDKVGLQERVEWVIENEAAILASAENPYDNRFWATAEKPWQALAFCFEWAGFKREGFAFHSSLPVQMDGTCNGLQNFSAMLLDEIGGRAVNLVPGEKPNDIYQTVADVVVEKLQQIAAACPEDTTEVETKDGGTRTVDSEGALARKWLAYGVTRKVTKRPVMTLAYGATKFGFRQQVFDDIVSEWRAEAKDAFPFEGAGWEAARFMGDLIWQCVGEVVVAARGAMDWLQEVAKIASKQRLPVIWTAPTGFKVMQEYTTAEQKRLELTFQKVRLRLQIDTSTKKLDVRKQSSGISPNWVHSMDAAHMQRTVARCHAEGIRSFSLIHDSYGTHAGNAWAMAQILREEFVRMYSEHDVLAEFREEIAMLLGVEPEALPKLPPKGSLDLHQVLESDFFFA

SEQ ID NO: 7 - protein, 858 aa, synthetic construct
GTENLTDITFFNTHGIKPSDHPMWEQQLELEAEMRSAGIARFRSMLEKSEQRGMMTGVASVRRLMVHAHAKTVEAFHAFMAEVKSSKAGRRHVAAAYLADADPDVACHLTLRALLDTVSQQMKLANAASRVATLIEDELHFNAFREQNEGAYNWSRRAILERSQNRNYQRRVMNKLARNRQIQWEDWTDEIKVRVGTKMIELVVEATGLFELRRMSDGRNFTNIYVTATQETLDWIKTENSRLEPMSPIYLPCLMPPRPWTSPVSGGYWSGRVRNLRLVKTHNRAYLEDLENADMQTVYDAVNAMQNTAWQINRPVYEVMRTLWDNQSTLGNLPMSEDEPLPEAPLWLEPGMTKEDMTPDQLEQFMRWKAERAAIYETNAKAVSKRLQFSRMLWVAERFKDVEEFYFPYQLDFRGRVYAVPLFLNPQGNDAARGLLTFANTVPITDQDGADWLAIHGASLWGVDKVGLQERVEWVIENEAAILASAENPYDNRFWATAEKPWQALAFCFEWAGFKREGFAFHSSLPVQMDGTCNGLQNFSAMLLDEIGGRAVNLVPGEKPNDIYQTVADVVVEKLQQIAAACPEDTTEVETKDGGTRTVDSEGALARKWLAYGVTRKVTKRPVMTLAYGATKFGFRQQVFDDIVSEWRAEAKDAFPFEGAGWEAARFMGDLIWQCVGEVVVAARGAMDWLQEVAKIASKQRLPVIWTAPTGFKVMQEYTTAEQKRLELTFQKVRLRLQIDTSTKKLDVRKQSSGISPNWVHSMDAAHMQRTVARCHAEGIRSFSLIHDSYGTHAGNAWAMAQFLREEFVRMYSEHDVLAEFREEIAMLLGVEPEALPKLPPKGSLDLKQVLESDFFFA

SEQ ID NO: 8 - protein, 858 aa, synthetic construct
GTENLTDITFFNTHGIKPSDHPMWEQQLELEAEMRSAGIARFRSMLEKSEQRGMMTGVASVRRLMVHAHAKTVEAFHAFMAEVKSSKAGRRHVAAAYLADADPDVACHLTLRALLDTVSQQMKLANAASRVATLIEDELHFNAFREQNEGAYNWSRRAILERSQNRNYQRRVMNKLARNRQIQWEDWTDEIKVRVGTKMIELVVEATGLFELRRISDGRNFTNIYVTATQETLDWIKTENSRLEPMSPIYLPCLMPPRPWTSPVSGGYWSGRVRNLRLVKTHNRAYLEDLENADMQTVYDAVNAMQNTAWQINRPVYEVMRTLWDNQSTLGNLPMSEDEPLPEAPLWLEPGMTKEDMTPDQLEQFMRWKAERAAIYETNAKAVSKRLQFSRMLWVAERFKDVEEFYFPYQLDFRGRVYAVPLFLNPQGNDAARGLLTFANTVPITDQDGADWLAIHGANLWGVDKVGLQERVEWVIENEAAILASAENPYDNRFWATAEKPWQALAFCFEWAGFKREGFAFHSSLPVQMDGTCNGLQHFSAMLLDEIGGRAVNLVPGEKPNDIYQTVADVVVEKLQQIAAACPEDTTEVETKDGGTRTVDSEGALARKWLAYGVTRKVTKRPVMTLAYGATKFGFRQQVFDDIVSEWRAEAKDAFPFEGAGWEAARFMGDLIWQCVGEVVVAARGAMDWLQEVAKIASKQRLPVIWTAPTGFKVMQEYTTAEQKRLELTFQKVRLRLQIDTSTKKLDVRKQSSGISPNFVHSMDAAHMQRTVARCHAEGIRSFSLIHDSYGTHAGNADAMAQILREEFVRMYSEHDVLAEFREEIAMLLGVEPEALPKLPPKGSLDLKQVLESDFFFA

SEQ ID NO: 9 - protein, 858 aa, synthetic construct
GTENLTDITFFNTHGIKPSDHPMWEQQLELEAEMRSAGIARFRSMLEKSEQRGMMTGVASVRRLMVHAHAKTVEAFHAFMAEVKSSKAGRRHVAAAYLADADPDVLCDLTLRALLDTVSQQMKLANAASRVATRIEDELHFNAFREQNEGAYNWSRRAILERSQNRNYQRRVMNKLARNRQIQWEDWTDEIKVRVGTKMIELVVEATGLFELRRISDGRNFTNIYVTATQEVLDWIKTENSRLEPMSPIYLPCLMPPRPWTSPVSGGYWSGRVRNLRLVKTHERAYLEDLENADMQTVYDAVNAMQNTAWQINRPVYEVMRTLWDNDSILGNLPMSEDEPLPEAPLWLEPGMTKEDMTPDQLEQFMRWKAERAAIYEKNAKAVSKRLQFSRMLWVAERFKDQEEFYFPYQLDFRGRVYPVPLFLNPQGNDAARGLLTFANTVPITDQDGADWLKIHGANLWGVDKVGLQERVEWVIENEDDILDSAENPLDNRFWATAEKPWQALAFCFEWAGFKEEGSAFHSSLPVQMDGTCNGLQHFSAMLLDEIGGRAVNLVPGEKPNDIYQTVADVVVEKLQQIAAACPEDTTEVETKDGGTRTVDSEGALARKWLAYGVTRKVTKRPVMTLAYGATKFGFRQQVFDDIVSEWRAEAKDAFPFEGAGWEAARFMGDLIWQCVGEVVVAARGAMDWLQEVAKIASKQRLPVIWTAPTGFKVWQPYTTAEQKRLELTFQKVRLRLQIDTSTKKLDVRKQSSGIPPNFVHSMDAAHMQRTVARCHAEGIRSFSLIHDSYGTHAGNADAMAQILREEFVRMYSEHDVLAEFREEIAMLLGVEPEALPKLPPKGSLDLKQVLESDFFFA

SEQ ID NO: 10 - protein, 858 aa, synthetic construct
GTENLTDITFFNTHGIKPSDHPMWEQQLELEAEMRSAGIARFRSMLEKSEQRGMMTGVASVRRLMVHAHAKTVEAFHAFMAEVKSSKAGRRHVAAAYLADADPDVLCDLTLRALLDTVSQQMKLANAASRVATRIEDELHFNAFREQNEGAYNWSRRAILERSQNRNYQRRVMNKLARNRQIQWEDWTDEIKVRVGTKMIELVVEATGLFELRRISDGRNFTNIYVTATQEVLDWIKTENSRLEPMSPIYLPCLMPPRPWTSPVSGGYWSGRVRNLRLVKTHERAYLEDLENADMQTVYDAVNAMQNTAWQINRPVYEVMRTLWDNDSILGNLPMSEDEPLPEAPLWLEPGMTKEDMTPDQLEQFMRWKAERAAIYEKNAKAVSKRLQFSRMLWVAERFKDSEEFYFPYQLDFRGRVYPVPLFLNPQGNDAARGLLTFANTVPITDQDGADWLKIHGANLWGVDKVGLQERVEWVIENEDDILDSAENPLDNRFWATAEKPWQALAFCFEWAGFKEEGSAFHSSLPVQMDGTCNGLQHFSAMLLDEIGGRAVNLVPGEKPNDIYQTVADVVVEKLQQIAAACPEDTTEVETKDGGTRTVDSEGALARKWLAYGVTRKVTKRPVMTLAYGATKFGFRQQVFDDIVSEWRAEAKDAFPFEGAGWEAARFMGDLIWQCVGEVVVAARGAMDWLQEVAKIASKQRLPVIWTAPTGFKVWQPYTTAEQKRLELTFQKVRLRLQIDTSTKKLDVRKQSSGIPPNFVHSMDAAHMQRTVARCHAEGIRSFSLIHDSYGTHAGNADAMAQILREEFVRMYSEHDVLAEFREEIAMLLGVEPEALPKLPPKGSLDLKQVLESDFFFA

SEQ ID NO: 11 - protein, 874 aa, synthetic construct
MGKIEESKHHHHHHGSGTENLTDITFFNTHGIKPSDHPMWEQQLELEAEMRSAGIARFRSMLEKSEQRGMMTGVASVRRLMVHAHAKTVEAFHAFMAEVKSSKAGRRHVAAAYLADADPDVACHLTLRALLDTVSQQMKLANAASRVATLIEDELHFNAFREQNEGAYNWSRRAILERSQNRNYQRRVMNKLARNRQIQWEDWTDEIKVRVGTKMIELVVEATGLFELRRISDGRNFTNIYVTATQETLDWIKTENSRLEPMSPIYLPCLMPPRPWTSPVSGGYWSGRVRNLRLVKTHNRAYLEDLENADMQTVYDAVNAMQNTAWQINRPVYEVMRTLWDNQSTLGNLPMSEDEPLPEAPLWLEPGMTKEDMTPDQLEQFMRWKAERAAIYETNAKAVSKRLQFSRMLWVAERFKDVEEFYFPYQLDFRGRVYAVPLFLNPQGNDAARGLLTFANTVPITDQDGADWLAIHGASLWGVDKVGLQERVEWVIENEAAILASAENPYDNRFWATAEKPWQALAFCFEWAGFKREGFAFHSSLPVQMDGTCNGLQNFSAMLLDEIGGRAVNLVPGEKPNDIYQTVADVVVEKLQQIAAACPEDTTEVETKDGGTRTVDSEGALARKWLAYGVTRKVTKRPVMTLAYGATKFGFRQQVFDDIVSEWRAEAKDAFPFEGAGWEAARFMGDLIWQCVGEVVVAARGAMDWLQEVAKIASKQRLPVIWTAPTGFKVMQEYTTAEQKRLELTFQKVRLRLQIDTSTKKLDVRKQSSGISPNWVHSMDAAHMQRTVARCHAEGIRSFSLIHDSYGTHAGNAWAMAQFLREEFVRMYSEHDVLAEFREEIAMLLGVEPEALPKLPPKGSLDLHQVLESDFFFA

SEQ ID NO: 12 - protein, 874 aa, synthetic construct
MGKIEESKHHHHHHGSGTENLTDITFFNTHGIKPSDHPMWEQQLELEAEMRSAGIARFRSMLEKSEQRGMMTGVASVRRLMVHAHAKTVEAFHAFMAEVKSSKAGRRHVAAAYLADADPDVACHLTLRALLDTVSQQMKLANAASRVATLIEDELHFNAFREQNEGAYNWSRRAILERSQNRNYQRRVMNKLARNRQIQWEDWTDEIKVRVGTKMIELVVEATGLFELRRMSDGRNFTNIYVTATQETLDWIKTENSRLEPMSPIYLPCLMPPRPWTSPVSGGYWSGRVRNLRLVKTHNRAYLEDLENADMQTVYDAVNAMQNTAWQINRPVYEVMRTLWDNQSTLGNLPMSEDEPLPEAPLWLEPGMTKEDMTPDQLEQFMRWKAERAAIYETNAKAVSKRLQFSRMLWVAERFKDVEEFYFPYQLDFRGRVYAVPLFLNPQGNDAARGLLTFANTVPITDQDGADWLAIHGANLWGVDKVGLQERVEWVIENEAAILASAENPYDNRFWATAEKPWQALAFCFEWAGFKREGFAFHSSLPVQMDGTCNGLQNFSAMLLDEIGGRAVNLVPGEKPNDIYQTVADVVVEKLQQIAAACPEDTTEVETKDGGTRTVDSEGALARKWLAYGVTRKVTKRPVMTLAYGATKFGFRQQVFDDIVSEWRAEAKDAFPFEGAGWEAARFMGDLIWQCVGEVVVAARGAMDWLQEVAKIASKQRLPVIWTAPTGFKVMQEYTTAEQKRLELTFQKVRLRLQIDTSTKKLDVRKQSSGISPNWVHSMDAAHMQRTVARCHAEGIRSFSLIHDSYGTHAGNAWAMAQFLREEFVRMYSEHDVLAEFREEIAMLLGVEPEALPKLPPKGSLDLHQVLESDFFFA

SEQ ID NO: 13 - protein, 874 aa, synthetic construct
MGKIEESKHHHHHHGSGTENLTDITFFNTHGIKPSDHPMWEQQLELEAEMRSAGIARFRSMLEKSEQRGMMTGVASVRRLMVHAHAKTVEAFHAFMAEVKSSKAGRRHVAAAYLADADPDVACHLTLRALLDTVSQQMKLANAASRVATLIEDELHFNAFREQNEGAYNWSRRAILERSQNRNYQRRVMNKLARNRQIQWEDWTDEIKVRVGTKMIELVVEATGLFELRRMSDGRNFTNIYVTATQETLDWIKTENSRLEPMSPIYLPCLMPPRPWTSPVSGGYWSGRVRNLRLVKTHNRAYLEDLENADMQTVYDAVNAMQNTAWQINRPVYEVMRTLWDNQSTLGNLPMSEDEPLPEAPLWLEPGMTKEDMTPDQLEQFMRWKAERAAIYETNAKAVSKRLQFSRMLWVAERFKDVEEFYFPYQLDFRGRVYAVPLFLNPQGNDAARGLLTFANTVPITDQDGADWLAIHGASLWGVDKVGLQERVEWVIENEAAILASAENPYDNRFWATAEKPWQALAFCFEWAGFKREGFAFHSSLPVQMDGTCNGLQHFSAMLLDEIGGRAVNLVPGEKPNDIYQTVADVVVEKLQQIAAACPEDTTEVETKDGGTRTVDSEGALARKWLAYGVTRKVTKRPVMTLAYGATKFGFRQQVFDDIVSEWRAEAKDAFPFEGAGWEAARFMGDLIWQCVGEVVVAARGAMDWLQEVAKIASKQRLPVIWTAPTGFKVMQEYTTAEQKRLELTFQKVRLRLQIDTSTKKLDVRKQSSGISPNWVHSMDAAHMQRTVARCHAEGIRSFSLIHDSYGTHAGNAWAMAQFLREEFVRMYSEHDVLAEFREEIAMLLGVEPEALPKLPPKGSLDLHQVLESDFFFA

SEQ ID NO: 14 - protein, 874 aa, synthetic construct
MGKIEESKHHHHHHGSGTENLTDITFFNTHGIKPSDHPMWEQQLELEAEMRSAGIARFRSMLEKSEQRGMMTGVASVRRLMVHAHAKTVEAFHAFMAEVKSSKAGRRHVAAAYLADADPDVACHLTLRALLDTVSQQMKLANAASRVATLIEDELHFNAFREQNEGAYNWSRRAILERSQNRNYQRRVMNKLARNRQIQWEDWTDEIKVRVGTKMIELVVEATGLFELRRMSDGRNFTNIYVTATQETLDWIKTENSRLEPMSPIYLPCLMPPRPWTSPVSGGYWSGRVRNLRLVKTHNRAYLEDLENADMQTVYDAVNAMQNTAWQINRPVYEVMRTLWDNQSTLGNLPMSEDEPLPEAPLWLEPGMTKEDMTPDQLEQFMRWKAERAAIYETNAKAVSKRLQFSRMLWVAERFKDVEEFYFPYQLDFRGRVYAVPLFLNPQGNDAARGLLTFANTVPITDQDGADWLAIHGASLWGVDKVGLQERVEWVIENEAAILASAENPYDNRFWATAEKPWQALAFCFEWAGFKREGFAFHSSLPVQMDGTCNGLQNFSAMLLDEIGGRAVNLVPGEKPNDIYQTVADVVVEKLQQIAAACPEDTTEVETKDGGTRTVDSEGALARKWLAYGVTRKVTKRPVMTLAYGATKFGFRQQVFDDIVSEWRAEAKDAFPFEGAGWEAARFMGDLIWQCVGEVVVAARGAMDWLQEVAKIASKQRLPVIWTAPTGFKVMQEYTTAEQKRLELTFQKVRLRLQIDTSTKKLDVRKQSSGISPNFVHSMDAAHMQRTVARCHAEGIRSFSLIHDSYGTHAGNAWAMAQFLREEFVRMYSEHDVLAEFREEIAMLLGVEPEALPKLPPKGSLDLHQVLESDFFFA

SEQ ID NO: 15 - protein, 874 aa, synthetic construct
MGKIEESKHHHHHHGSGTENLTDITFFNTHGIKPSDHPMWEQQLELEAEMRSAGIARFRSMLEKSEQRGMMTGVASVRRLMVHAHAKTVEAFHAFMAEVKSSKAGRRHVAAAYLADADPDVACHLTLRALLDTVSQQMKLANAASRVATLIEDELHFNAFREQNEGAYNWSRRAILERSQNRNYQRRVMNKLARNRQIQWEDWTDEIKVRVGTKMIELVVEATGLFELRRMSDGRNFTNIYVTATQETLDWIKTENSRLEPMSPIYLPCLMPPRPWTSPVSGGYWSGRVRNLRLVKTHNRAYLEDLENADMQTVYDAVNAMQNTAWQINRPVYEVMRTLWDNQSTLGNLPMSEDEPLPEAPLWLEPGMTKEDMTPDQLEQFMRWKAERAAIYETNAKAVSKRLQFSRMLWVAERFKDVEEFYFPYQLDFRGRVYAVPLFLNPQGNDAARGLLTFANTVPITDQDGADWLAIHGASLWGVDKVGLQERVEWVIENEAAILASAENPYDNRFWATAEKPWQALAFCFEWAGFKREGFAFHSSLPVQMDGTCNGLQNFSAMLLDEIGGRAVNLVPGEKPNDIYQTVADVVVEKLQQIAAACPEDTTEVETKDGGTRTVDSEGALARKWLAYGVTRKVTKRPVMTLAYGATKFGFRQQVFDDIVSEWRAEAKDAFPFEGAGWEAARFMGDLIWQCVGEVVVAARGAMDWLQEVAKIASKQRLPVIWTAPTGFKVMQEYTTAEQKRLELTFQKVRLRLQIDTSTKKLDVRKQSSGISPNWVHSMDAAHMQRTVARCHAEGIRSFSLIHDSYGTHAGNADAMAQFLREEFVRMYSEHDVLAEFREEIAMLLGVEPEALPKLPPKGSLDLHQVLESDFFFA

SEQ ID NO: 16 - protein, 874 aa, synthetic construct
MGKIEESKHHHHHHGSGTENLTDITFFNTHGIKPSDHPMWEQQLELEAEMRSAGIARFRSMLEKSEQRGMMTGVASVRRLMVHAHAKTVEAFHAFMAEVKSSKAGRRHVAAAYLADADPDVACHLTLRALLDTVSQQMKLANAASRVATLIEDELHFNAFREQNEGAYNWSRRAILERSQNRNYQRRVMNKLARNRQIQWEDWTDEIKVRVGTKMIELVVEATGLFELRRMSDGRNFTNIYVTATQETLDWIKTENSRLEPMSPIYLPCLMPPRPWTSPVSGGYWSGRVRNLRLVKTHNRAYLEDLENADMQTVYDAVNAMQNTAWQINRPVYEVMRTLWDNQSTLGNLPMSEDEPLPEAPLWLEPGMTKEDMTPDQLEQFMRWKAERAAIYETNAKAVSKRLQFSRMLWVAERFKDVEEFYFPYQLDFRGRVYAVPLFLNPQGNDAARGLLTFANTVPITDQDGADWLAIHGASLWGVDKVGLQERVEWVIENEAAILASAENPYDNRFWATAEKPWQALAFCFEWAGFKREGFAFHSSLPVQMDGTCNGLQNFSAMLLDEIGGRAVNLVPGEKPNDIYQTVADVVVEKLQQIAAACPEDTTEVETKDGGTRTVDSEGALARKWLAYGVTRKVTKRPVMTLAYGATKFGFRQQVFDDIVSEWRAEAKDAFPFEGAGWEAARFMGDLIWQCVGEVVVAARGAMDWLQEVAKIASKQRLPVIWTAPTGFKVMQEYTTAEQKRLELTFQKVRLRLQIDTSTKKLDVRKQSSGISPNWVHSMDAAHMQRTVARCHAEGIRSFSLIHDSYGTHAGNAWAMAQILREEFVRMYSEHDVLAEFREEIAMLLGVEPEALPKLPPKGSLDLHQVLESDFFFA

SEQ ID NO: 17 - protein, 874 aa, synthetic construct
MGKIEESKHHHHHHGSGTENLTDITFFNTHGIKPSDHPMWEQQLELEAEMRSAGIARFRSMLEKSEQRGMMTGVASVRRLMVHAHAKTVEAFHAFMAEVKSSKAGRRHVAAAYLADADPDVACHLTLRALLDTVSQQMKLANAASRVATLIEDELHFNAFREQNEGAYNWSRRAILERSQNRNYQRRVMNKLARNRQIQWEDWTDEIKVRVGTKMIELVVEATGLFELRRMSDGRNFTNIYVTATQETLDWIKTENSRLEPMSPIYLPCLMPPRPWTSPVSGGYWSGRVRNLRLVKTHNRAYLEDLENADMQTVYDAVNAMQNTAWQINRPVYEVMRTLWDNQSTLGNLPMSEDEPLPEAPLWLEPGMTKEDMTPDQLEQFMRWKAERAAIYETNAKAVSKRLQFSRMLWVAERFKDVEEFYFPYQLDFRGRVYAVPLFLNPQGNDAARGLLTFANTVPITDQDGADWLAIHGASLWGVDKVGLQERVEWVIENEAAILASAENPYDNRFWATAEKPWQALAFCFEWAGFKREGFAFHSSLPVQMDGTCNGLQNFSAMLLDEIGGRAVNLVPGEKPNDIYQTVADVVVEKLQQIAAACPEDTTEVETKDGGTRTVDSEGALARKWLAYGVTRKVTKRPVMTLAYGATKFGFRQQVFDDIVSEWRAEAKDAFPFEGAGWEAARFMGDLIWQCVGEVVVAARGAMDWLQEVAKIASKQRLPVIWTAPTGFKVMQEYTTAEQKRLELTFQKVRLRLQIDTSTKKLDVRKQSSGISPNWVHSMDAAHMQRTVARCHAEGIRSFSLIHDSYGTHAGNAWAMAQFLREEFVRMYSEHDVLAEFREEIAMLLGVEPEALPKLPPKGSLDLKQVLESDFFFA

SEQ ID NO: 18 - protein, 874 aa, synthetic construct
MGKIEESKHHHHHHGSGTENLTDITFFNTHGIKPSDHPMWEQQLELEAEMRSAGIARFRSMLEKSEQRGMMTGVASVRRLMVHAHAKTVEAFHAFMAEVKSSKAGRRHVAAAYLADADPDVACHLTLRALLDTVSQQMKLANAASRVATLIEDELHFNAFREQNEGAYNWSRRAILERSQNRNYQRRVMNKLARNRQIQWEDWTDEIKVRVGTKMIELVVEATGLFELRRISDGRNFTNIYVTATQETLDWIKTENSRLEPMSPIYLPCLMPPRPWTSPVSGGYWSGRVRNLRLVKTHNRAYLEDLENADMQTVYDAVNAMQNTAWQINRPVYEVMRTLWDNQSTLGNLPMSEDEPLPEAPLWLEPGMTKEDMTPDQLEQFMRWKAERAAIYETNAKAVSKRLQFSRMLWVAERFKDVEEFYFPYQLDFRGRVYAVPLFLNPQGNDAARGLLTFANTVPITDQDGADWLAIHGANLWGVDKVGLQERVEWVIENEAAILASAENPYDNRFWATAEKPWQALAFCFEWAGFKREGFAFHSSLPVQMDGTCNGLQHFSAMLLDEIGGRAVNLVPGEKPNDIYQTVADVVVEKLQQIAAACPEDTTEVETKDGGTRTVDSEGALARKWLAYGVTRKVTKRPVMTLAYGATKFGFRQQVFDDIVSEWRAEAKDAFPFEGAGWEAARFMGDLIWQCVGEVVVAARGAMDWLQEVAKIASKQRLPVIWTAPTGFKVMQEYTTAEQKRLELTFQKVRLRLQIDTSTKKLDVRKQSSGISPNFVHSMDAAHMQRTVARCHAEGIRSFSLIHDSYGTHAGNADAMAQILREEFVRMYSEHDVLAEFREEIAMLLGVEPEALPKLPPKGSLDLKQVLESDFFFA

SEQ ID NO: 19 - protein, 874 aa, synthetic construct
MGKIEESKHHHHHHGSGTENLTDITFFNTHGIKPSDHPMWEQQLELEAEMRSAGIARFRSMLEKSEQRGMMTGVASVRRLMVHAHAKTVEAFHAFMAEVKSSKAGRRHVAAAYLADADPDVLCDLTLRALLDTVSQQMKLANAASRVATRIEDELHFNAFREQNEGAYNWSRRAILERSQNRNYQRRVMNKLARNRQIQWEDWTDEIKVRVGTKMIELVVEATGLFELRRISDGRNFTNIYVTATQEVLDWIKTENSRLEPMSPIYLPCLMPPRPWTSPVSGGYWSGRVRNLRLVKTHERAYLEDLENADMQTVYDAVNAMQNTAWQINRPVYEVMRTLWDNDSILGNLPMSEDEPLPEAPLWLEPGMTKEDMTPDQLEQFMRWKAERAAIYEKNAKAVSKRLQFSRMLWVAERFKDQEEFYFPYQLDFRGRVYPVPLFLNPQGNDAARGLLTFANTVPITDQDGADWLKIHGANLWGVDKVGLQERVEWVIENEDDILDSAENPLDNRFWATAEKPWQALAFCFEWAGFKEEGSAFHSSLPVQMDGTCNGLQHFSAMLLDEIGGRAVNLVPGEKPNDIYQTVADVVVEKLQQIAAACPEDTTEVETKDGGTRTVDSEGALARKWLAYGVTRKVTKRPVMTLAYGATKFGFRQQVFDDIVSEWRAEAKDAFPFEGAGWEAARFMGDLIWQCVGEVVVAARGAMDWLQEVAKIASKQRLPVIWTAPTGFKVWQPYTTAEQKRLELTFQKVRLRLQIDTSTKKLDVRKQSSGIPPNFVHSMDAAHMQRTVARCHAEGIRSFSLIHDSYGTHAGNADAMAQILREEFVRMYSEHDVLAEFREEIAMLLGVEPEALPKLPPKGSLDLKQVLESDFFFA

SEQ ID NO: 20 - protein, 874 aa, synthetic construct
MGKIEESKHHHHHHGSGTENLTDITFFNTHGIKPSDHPMWEQQLELEAEMRSAGIARFRSMLEKSEQRGMMTGVASVRRLMVHAHAKTVEAFHAFMAEVKSSKAGRRHVAAAYLADADPDVLCDLTLRALLDTVSQQMKLANAASRVATRIEDELHFNAFREQNEGAYNWSRRAILERSQNRNYQRRVMNKLARNRQIQWEDWTDEIKVRVGTKMIELVVEATGLFELRRISDGRNFTNIYVTATQEVLDWIKTENSRLEPMSPIYLPCLMPPRPWTSPVSGGYWSGRVRNLRLVKTHERAYLEDLENADMQTVYDAVNAMQNTAWQINRPVYEVMRTLWDNDSILGNLPMSEDEPLPEAPLWLEPGMTKEDMTPDQLEQFMRWKAERAAIYEKNAKAVSKRLQFSRMLWVAERFKDSEEFYFPYQLDFRGRVYPVPLFLNPQGNDAARGLLTFANTVPITDQDGADWLKIHGANLWGVDKVGLQERVEWVIENEDDILDSAENPLDNRFWATAEKPWQALAFCFEWAGFKEEGSAFHSSLPVQMDGTCNGLQHFSAMLLDEIGGRAVNLVPGEKPNDIYQTVADVVVEKLQQIAAACPEDTTEVETKDGGTRTVDSEGALARKWLAYGVTRKVTKRPVMTLAYGATKFGFRQQVFDDIVSEWRAEAKDAFPFEGAGWEAARFMGDLIWQCVGEVVVAARGAMDWLQEVAKIASKQRLPVIWTAPTGFKVWQPYTTAEQKRLELTFQKVRLRLQIDTSTKKLDVRKQSSGIPPNFVHSMDAAHMQRTVARCHAEGIRSFSLIHDSYGTHAGNADAMAQILREEFVRMYSEHDVLAEFREEIAMLLGVEPEALPKLPPKGSLDLKQVLESDFFFA

SEQ ID NO: 21 - protein, 858 aa, synthetic construct
GTENLTDITFFNTHGIKPSDHPMWEQQLELEAEMRSAGIARFRSMLEKSEQRGMMTGVASVRRLMVHAHAKTVEAFHAFMAEVKSSKAGRRHVAAAYLADADPDVACHLTLRALLDTVSQQMKLANAASRVATLIEDELHFNAFREQNEGAYNWSRRAILERSQNRNYQRRVMNKLARNRQIQWEDWTDEIKVRVGTKMIELVVEATGLFELRRMSDGRNFTNIYVTATQETLDWIKTENSRLEPMSPIYLPCLMPPRPWTSPVSGGYWSGRVRNLRLVKTHNRAYLEDLENADMQTVYDAVNAMQNTAWQINRPVYEVMRTLWDNQSTLGNLPMSEDEPLPEAPLWLEPGMTKEDMTPDQLEQFMRWKAERAAIYETNAKAVSKRLQFSRMLWVAERFKDVEEFYFPYQLDFRGRVYAVPLFLNPQGNDAARGLLTFANTVPITDQDGADWLAIHGASLWGVDKVGLQERVEWVIENEAAILASAENPYDNRFWATAEKPWQALAFCFEWAGFKREGFAFHSSLPVQMDGTCNGLQNFSAMLLDEIGGRAVNLVPGEKPNDIYQTVADVVVEKLQQIAAACPEDTTEVETKDGGTRTVDSEGALARKWLAYGVTRKVTKRPVMTLAYGATKFGFRQQVFDDIVSEWRAEAKDAFPFEGAGWEAARFMGDLIWQCVGEVVVAARGAMDWLQEVAKIASKQRLPVIWTAPTGFKVMQEYTTAEQKRLELTFQKVRLRLQIDTSTKKLDVRKQSSGISPNWVHSMDAAHMQRTVARCHAEGIRSFSLIHDSYGTHAGNAWAMAQFLREEFVRMYSEHDVLAEFREEIAMLLGVEPEALPKLPPKGSLDLHQVLESDFFFA

SEQ ID NO: 22 - protein, 873 aa, synthetic construct
MKIEESKHHHHHHGSGTENLTDITFFNTHGIKPSDHPMWEQQLELEAEMRSAGIARFRSMLEKSEQRGMMTGVASVRRLMVHAHAKTVEAFHAFMAEVKSSKAGRRHVAAAYLADADPDVACHLTLRALLDTVSQQMKLANAASRVATLIEDELHFNAFREQNEGAYNWSRRAILERSQNRNYQRRVMNKLARNRQIQWEDWTDEIKVRVGTKMIELVVEATGLFELRRMSDGRNFTNIYVTATQETLDWIKTENSRLEPMSPIYLPCLMPPRPWTSPVSGGYWSGRVRNLRLVKTHNRAYLEDLENADMQTVYDAVNAMQNTAWQINRPVYEVMRTLWDNQSTLGNLPMSEDEPLPEAPLWLEPGMTKEDMTPDQLEQFMRWKAERAAIYETNAKAVSKRLQFSRMLWVAERFKDVEEFYFPYQLDFRGRVYAVPLFLNPQGNDAARGLLTFANTVPITDQDGADWLAIHGASLWGVDKVGLQERVEWVIENEAAILASAENPYDNRFWATAEKPWQALAFCFEWAGFKREGFAFHSSLPVQMDGTCNGLQNFSAMLLDEIGGRAVNLVPGEKPNDIYQTVADVVVEKLQQIAAACPEDTTEVETKDGGTRTVDSEGALARKWLAYGVTRKVTKRPVMTLAYGATKFGFRQQVFDDIVSEWRAEAKDAFPFEGAGWEAARFMGDLIWQCVGEVVVAARGAMDWLQEVAKIASKQRLPVIWTAPTGFKVMQEYTTAEQKRLELTFQKVRLRLQIDTSTKKLDVRKQSSGISPNWVHSMDAAHMQRTVARCHAEGIRSFSLIHDSYGTHAGNAWAMAQFLREEFVRMYSEHDVLAEFREEIAMLLGVEPEALPKLPPKGSLDLHQVLESDFFFA

SEQ ID NO: 23 - protein, 17 aa, synthetic construct
RATTACCMCCCACAWKA

SEQ ID NO: 24 - RNA, 17 nt, synthetic construct
  source 1..17: mol_type = other RNA
aattaccccccacaata

SEQ ID NO: 25 - RNA, 17 nt, synthetic construct
  source 1..17: mol_type = other RNA
gattaccacccacaata

SEQ ID NO: 26 - RNA, 17 nt, synthetic construct
  source 1..17: mol_type = other RNA
aattaccccccacaaga

SEQ ID NO: 27 - RNA, 17 nt, synthetic construct
  source 1..17: mol_type = other RNA
aattaccccccactata

SEQ ID NO: 28 - protein, 883 aa, synthetic construct
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 29 - RNA, 17 nt, synthetic construct
  source 1..17: mol_type = other RNA
taatacgactcactata

SEQ ID NO: 30 - protein, 822 aa, synthetic construct
MISALSTVVVPEEALVKRQLELEETYKIRGIERARKLITDALQNGGIMNLPMTQRMLTSAYEVAAAAIDEMRNVKAPGIGGKYRRFLRLIPLDVLTTLSLCTMFEAFSVAPGESASRRQTAQAVMSALGRNVQSELLSLQLRNVAPAYMDRVYEYLTERRTKSPTHILRTLRASAENVHYGHEPWTNAQNISVGRLLCAAVFETGLFQWKTGSGNLSMLYPADDVMEAFQQLVESADTVTMKPPMLVPPVQHTTMWDGGYLTPIDNRGTYHNSHIDRTRLREVAEAFKSADGIKKALNKAQETPYRINKRILELVQEARALGIGVGMPRSVPEPKPEWYLDGVPKENYTEEELDRFGEWKTRMSLWYSADRKRVSQLRSLLTTLEMAEEFKDEKALYFPTCVDWRYRLYFKSSLHPQGSDLQKALLEFGRGKPLGDRGLFWLKVHVATCFGYDKTLFEDRAAWVDANFAEIEQLTVSPFDCPAFTSADSPWCLLAAAIDLVNAVRSGCPEEHISRIPVAMDATNSGGQHLSALLRDPVGGRLTNLYWEGNDKKADLYMDVKRRTDEKVILDLDKEDFIIQSTYWRENEITRSMTKRPSMTYFYSATVRSCSDYIFEGACAEGYEGTDTNSLWNLSCYLAPRMRAAIEEANPAAAAVMGYLQNLARRVPASQHLQWYTPLGGLVMNRYTQREEVRVRIDCMNLSAVLVHNRDFKTCNKRKAASGIAPNFVHSLDSTHLMMVLCAAEGLDIVPIHDSLATHAADVDDMHRHIREQFVRLYEENDLLGDITRAAAAAGADLTDLDMPEVGTLDIRQVLESPFFFC

SEQ ID NO: 31 - RNA, 17 nt, synthetic construct
  source 1..17: mol_type = other RNA
atttaggtgacactata

SEQ ID NO: 32 - RNA, 17 nt, synthetic construct
  source 1..17: mol_type = other RNA
aattaaccctcactaaa

SEQ ID NO: 33 - RNA, 23 nt, synthetic construct
  source 1..23: mol_type = other RNA
gaataagtagaggtgaagattta

SEQ ID NO: 34 - RNA, 30 nt, synthetic construct
  source 1..30: mol_type = other RNA
gatgatgatgatgatgatgatgatgatgat

SEQ ID NO: 35 - RNA, 50 nt, synthetic construct
  source 1..50: mol_type = other RNA
gggaagcttcggctggtgcagtggcctcataagaggcggcccctaacagg

SEQ ID NO: 36 - RNA, 1122 nt, synthetic construct
  source 1..1122: mol_type = other RNA
gggtctagaaataattttgtttaactttagagtacacgagtcaggctacagcatcctctggttcagactacttgattcatgtgtaccctatatgcgaggatatgtgtatcgtagaaattgtcaggcagtaacgttccgcgagttttaatgggcgcgccatgactctaagagtgatatacctcctcggtctcgggcccggggtgtaattagcccagttagacacgatcgcccgacgtatattgttgcttgggtatcgtcgcatgcgaagtattgcccaaggagacacaacaagcaacttatgttgactcccttcgaccattaaaatttgttagaacggacagaaaggatgcgccttataaatgtcctgtgcagtgatgaagcgacctcaaaacgcttcatgatctaaccgactcaccttgccgttccctccgcgccttaaaaccggccggtcttgcgaaaagcgggaaacgagtttacccacggatagcagggaatgttgcggctggctagggagcatgaaggtagatactccacggcttacctttccggggctcaacatctagccacagaccttttcgttaagcccacccccactggatactgaatcatcagggaaccggacccaaccagtttgggctcgtccaagcttcggtctcgtccctaagtgcaaagatatggaaagagcagcataggtatatggattattcttttaccactcgtttcttaccgtaacttacgcaatggatcacgtgccgaggcggcggtacagctgttcgaagggctctgtcgggaacgctaacatccagccggtaaattccaaactagggaaaggacacgcactgaattgaatatagtcgtgaagggtggtgtaagtcgtgcacagcccgcattaagtactaaacagcgtccaatcttgatctacttacggcctgatgttcttcagcacctcctagcactggagtacttcgctatcaatgagattagcactttgtacatgtcatccagcccgagtctggggtccgacaatgcggtcgccgattggtatctgcatgtagtattaaacggagctgccgcggctgcggattatagttcatgtcttgacggtcctcgtgaactgtggtt

SEQ ID NO: 37 - RNA, 1707 nt, synthetic construct
  source 1..1707: mol_type = other RNA
gggagacccaagcttggtaccgagctcggatccgccaccatgaagaccttaattcttgccgttgcattagtctactgcgccactgttcattgccaggactgtccttacgaacctgatccaccaaacacagttccaacttcctgtgaagctaaagaaggagaatgtattgatagcagctgtggcacctgcacgagagacatactatcagatggactgtgtgaaaataaaccaggaaaaacatgttgccgaatgtgtcagtatgtaattgaatgcagagtagaggccgcaggatggtttagaacattctatggaaagagattccagttccaggaacctggtacatacgtgttgggtcaaggaaccaagggcggcgactggaaggtgtccatcaccctggagaacctggatggaaccaagggggctgtgctgaccaagacaagactggaagtggctggagacatcattgacatcgctcaagctactgagaatcccatcactgtaaacggtggagctgaccctatcatcgccaacccgtacaccatcggcgaggtcaccatcgctgttgttgagatgccaggcttcaacatcaccgtcattgagttcttcaaactgatcgtgatcgacatcctcggaggaagatctgtaagaatcgccccagacacagcaaacaaaggaatgatctctggcctctgtggagatcttaaaatgatggaagatacagacttcacttcagatccagaacaactcgctattcagcctaagatcaaccaggagtttgacggttgtccactctatggaaatcctgatgacgttgcatactgcaaaggtcttctggagccgtacaaggacagctgccgcaaccccatcaacttctactactacaccatctcctgcgccttcgcccgctgtatgggtggagacgagcgagcctcacacgtgctgcttgactacagggagacgtgcgctgctcccgaaactagaggaacctgcgttttgtctggacatactttctacgatacatttgacaaagcaagataccaattccagggtccctgcaaggagattcttatggccgccgactgtttctggaacacttgggatgtgaaggtttcacacaggaatgttgactcttacactgaagtagagaaagtacgaatcaggaaacaatcgactgtagtagaactcattgttgatggaaaacagattctggttggaggagaagccgtgtccgtcccgtacagctctcagaacacttccatctactggcaagatggtgacatactgactacagccatcctacctgaagctctggtggtcaagttcaacttcaagcaactgctcgtcgtacatattagagatccattcgatggtaagacttgcggtatttgcggtaactacaaccaggatttcagtgatgattcttttgatgctgaaggagcctgtgatctgacccccaacccaccgggatgcaccgaagaacagaaacctgaagctgaacgactctgcaatagtctcttcgccggtcaaagtgatcttgatcagaaatgtaacgtgtgccacaagcctgaccgtgtcgaacgatgcatgtacgagtattgcctgaggggacaacagggtttctgtgaccacgcatgggagttcaagaaagaatgctacataaagcatggagacaccctagaagtaccagatgaatgcaaataggcggcc

SEQ ID NO: 38 - RNA, 7783 nt, synthetic construct
  source 1..7783: mol_type = other RNA
taatacgactcactatagggaattgtgagcggataacaattcccctctagaaataattttgtttaactttaagaaggagatatacatatgaaaatcgaagaaagtaaacaccaccaccaccaccacggtagcggcaccgaaaacctgaccgatatcaccttctttaacacccacggtattaagccgagcgaccacccgatgtgggagcagcaactggagctggaagcggagatgcgtagcgcgggtattgcgcgtttccgtagcatgctggaaaagagcgagcagcgtggtatgatgaccggcgtggcgagcgttcgtcgtctgatggtgcacgcgcacgcgaaaaccgttgaagcgtttcacgcgttcatggcggaagtgaagagcagcaaagcgggtcgtcgtcatgttgcggcggcgtatctggcggatgcggatccggatgttgcgtgccacctgaccctgcgtgcgctgctggataccgtgagccagcaaatgaaactggcgaacgcggcgagccgtgttgcgaccctgattgaagacgagctgcactttaacgcgttccgtgaacagaacgagggtgcgtacaactggagccgtcgtgcgatcctggaacgtagccagaaccgtaactatcaacgtcgtgtgatgaacaaactggcgcgtaaccgtcagatccaatgggaagattggaccgacgagattaaagtgcgtgttggcaccaaaatgatcgaactggtggttgaggcgaccggtctgtttgaactgcgtcgtatgagcgatggccgtaacttcaccaacatttacgtgaccgcgacccaggaaaccctggactggatcaagaccgaaaacagccgtctggagccgatgagcccgatttacctgccgtgcctgatgccgccgcgtccgtggaccagcccggttagcggtggctattggagcggtcgtgtgcgtaacctgcgtctggttaaaacccacaaccgtgcgtacctggaagatctggagaacgcggacatgcagaccgtgtatgacgcggttaacgcgatgcagaacaccgcgtggcaaatcaaccgtccggtgtatgaggttatgcgtaccctgtgggataaccaaagcaccctgggtaacctgccgatgagcgaagatgagccgctgccggaagcgccgctgtggctggaaccgggtatgaccaaagaggacatgaccccggaccagctggaacaatttatgcgttggaaagcggaacgtgcggcgatttacgaaaccaacgcgaaggcggtgagcaaacgtctgcagtttagccgtatgctgtgggtggcggagcgtttcaaggatgttgaggaattttacttcccgtatcagctggactttcgtggtcgtgtgtatgcggttccgctgttcctgaacccgcaaggcaacgatgcggcgcgtggtctgctgaccttcgcgaacaccgttccgattaccgatcaagatggtgcggattggctggcgatccatggtgcgagcctgtggggcgtggacaaagttggtctgcaagaacgtgtggagtgggttattgaaaacgaggcggcgattctggcgagcgcggaaaacccgtacgacaaccgtttttgggcgaccgcggagaagccgtggcaggcgctggcgttttgcttcgaatgggcgggcttcaaacgtgagggttttgcgttccacagcagcctgccggtgcagatggatggtacctgcaacggcctgcaaaacttcagcgcgatgctgctggacgaaatcggtggccgtgcggtgaacctggttccgggcgagaagccgaacgatatttatcaaaccgttgcggacgtggttgtggaaaaactgcaacaaattgcggcggcgtgcccggaggataccaccgaagtggaaaccaaggatggtggcacccgtaccgttgacagcgagggtgcgctggcgcgtaaatggctggcgtacggcgtgacccgtaaggttaccaaacgtccggtgatgaccctggcgtatggcgcgaccaagtttggtttccgtcagcaagtgtttgacgatattgttagcgaatggcgtgcggaggcgaaagatgcgtttccgtttgagggtgcgggttgggaggcggcgcgtttcatgggcgacctgatctggcagtgcgtgggtgaagttgtggttgcggcgcgtggcgcgatggactggctgcaggaagtggcgaagattgcgagcaaacaacgtctgccggtgatctggaccgcgccgaccggttttaaggttatgcaggaatacaccaccgcggagcaaaagcgtctggaactgaccttccagaaagttcgtctgcgtctgcaaattgataccagcaccaagaaactggacgtgcgtaaacagagcagcggtatcagcccgaactgggttcacagcatggatgcggcgcacatgcaacgtaccgtggcgcgttgccatgcggaaggtatccgtagctttagcctgattcacgacagctacggtacccatgcgggtaacgcgtgggcgatggcgcagtttctgcgtgaggaattcgtgcgtatgtatagcgaacacgacgttctggcggagttccgtgaggaaatcgcgatgctgctgggtgtggaaccggaagcgctgccgaagctgccgccgaaaggcagcctggatctgcaccaagttctggagagcgacttctttttcgcgtaatagctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaat

SEQ ID NO: 39 - RNA, 3001 nt, synthetic construct
  source 1..3001: mol_type = other RNA
gcggcaattaccccccacaatagggtctagaaataattttgtttaactttaagaaggagatatacatatgaaaatcgaagaaggtaaaggtcaccatcaccatcaccacggatccatgtctaagggcgaggaactgtttacgggcgtcgttccgatcctggtggaactggatggcgacgtcaacggccataagttttctgtgtctggcgagggtgagggcgatgcgacctacggcaagttaacgctgaagctgatctgcaccaccggcaagctgccagtcccgtggccgaccctggtgacgacgctgggctacggtttacagtgcttcgcgcgctacccggatcacatgaagcagcacgactttttcaagagcgcgatgccggaaggctacgtccaggaacgcacgatcttcttcaaggacgacggcaactataagacgcgcgcagaggttaagttcgaaggcgacacgctggtcaaccgcatcgagctgaagggcattgacttcaaggaggacggcaacatcctgggccacaagctggagtacaactacaacagccacaacgtgtatatcaccgccgacaagcaaaaaaacggcatcaaggccaacttcaagatccgccacaacatcgaggacggtggcgtgcagctggccgatcactatcaacaaaacacgccgatcggtgacggtccggtcctgctgccggacaaccactacctgagctaccaaagcgccctgtctaaggacccgaacgagaagcgcgaccacatggtgttactggagtttgtcaccgcagccggcattacccacggtatggatgaactgtataaataactcgagaccggttaacctgcaggaggcctttaattaaggtggtgcggccgcgctagcggtcccgggggatcgatccggctgctaacaaagcccgaaaggaagctgagttggctgctgccaccgctgagcaataactagcataaccccttggggcctctaaacgggtcttgaggggttttttgctgaaaggaggaactatatccgggaagcttggcactggccgaccggggtcgagcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacagatccgggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgctagtggtgctagccccgcgaaat

SEQ ID NO: 40 - protein, 867 aa, synthetic construct
MHHHHHHGSGTENLTDITFFNTHGIKPSDHPMWEQQLELEAEMRSAGIARFRSMLEKSEQRGMMTGVASVRRLMVHAHAKTVEAFHAFMAEVKSSKAGRRHVAAAYLADADPDVACHLTLRALLDTVSQQMKLANAASRVATLIEDELHFNAFREQNEGAYNWSRRAILERSQNRNYQRRVMNKLARNRQIQWEDWTDEIKVRVGTKMIELVVEATGLFELRRMSDGRNFTNIYVTATQETLDWIKTENSRLEPMSPIYLPCLMPPRPWTSPVSGGYWSGRVRNLRLVKTHNRAYLEDLENADMQTVYDAVNAMQNTAWQINRPVYEVMRTLWDNQSTLGNLPMSEDEPLPEAPLWLEPGMTKEDMTPDQLEQFMRWKAERAAIYETNAKAVSKRLQFSRMLWVAERFKDVEEFYFPYQLDFRGRVYAVPLFLNPQGNDAARGLLTFANTVPITDQDGADWLAIHGASLWGVDKVGLQERVEWVIENEAAILASAENPYDNRFWATAEKPWQALAFCFEWAGFKREGFAFHSSLPVQMDGTCNGLQNFSAMLLDEIGGRAVNLVPGEKPNDIYQTVADVVVEKLQQIAAACPEDTTEVETKDGGTRTVDSEGALARKWLAYGVTRKVTKRPVMTLAYGATKFGFRQQVFDDIVSEWRAEAKDAFPFEGAGWEAARFMGDLIWQCVGEVVVAARGAMDWLQEVAKIASKQRLPVIWTAPTGFKVMQEYTTAEQKRLELTFQKVRLRLQIDTSTKKLDVRKQSSGISPNWVHSMDAAHMQRTVARCHAEGIRSFSLIHDSYGTHAGNAWAMAQFLREEFVRMYSEHDVLAEFREEIAMLLGVEPEALPKLPPKGSLDLHQVLESDFFFA

(19) United States
(12) Patent Application Publication
(10) Pub. No.: US 2026/0218263 A1
(43) Pub. Date: Jul. 30, 2026

(54) RNA POLYMERASES

(71) Applicant: New England Biolabs, Inc., Ipswich, MA (US)

(72) Inventors: Jackson A. Buss, Groveland, MA (US); Vladimir Potapov, Auburndale, MA (US); Peter R. Weigele, Cambridge, MA (US); Jennifer Ong, Salem, MA (US); Lili Mitchell, Ipswich, MA (US); Dillon B. Nye, Somerville, MA (US); Tien-Hao Chen, Wakefield, MA (US); G. B. Robb, Somerville, MA (US)

(73) Assignee: New England Biolabs, Inc., Ipswich, MA (US)

(21) Appl. No.: 19/564,565

(22) Filed: Mar. 12, 2026

Related U.S. Application Data
(63) Continuation of application No. PCT/US24/46670, filed on Sep. 13, 2024.
(60) Provisional application No. 63/582,625, filed on Sep. 14, 2023.

(51) Int. Cl. C12P 19/34; C12N 9/12; C12Q 1/6806

(52) U.S. Cl. CPC C12P 19/34; C12N 9/1247; C12Q 1/6806; C07K 2319/02; C12Y 207/07006

(57) ABSTRACT

The present disclosure relates, according to some embodiments, to RNA polymerases and fusions, compositions, kits, methods, and workflows including RNA polymerases. According to some embodiments, an RNA polymerase may include an H6 RNA polymerase (e.g., SEQ ID NO: 21) and/or variants thereof (e.g., variants having ≥90%, ≥95%, ≥98% to any of SEQ ID NOS: 1-20 and/or 22).

### CROSS-REFERENCE TO RELATED APPLICATIONS

This application is a continuation of International Application No. PCT/US2024/046670, filed Sep. 13, 2024, which claims the benefit of U.S. Provisional Application Ser. No. 63/582,625, filed Sep. 14, 2023. The contents of all of the above are hereby incorporated in their entirety by reference.

### SEQUENCE LISTING STATEMENT

This disclosure includes a Sequence Listing submitted electronically in .xml format under the file name “NEB-485” created on Sep. 13, 2024, and having a size of 70,812 bytes. This Sequence Listing is incorporated herein in its entirety by this reference.

### BACKGROUND

The potential for mRNA therapeutics is beginning to be appreciated. While mRNA manufacturing may be cell-free and scalable, challenges can arise where products include duplex RNA. Duplex RNA may be associated with heightened antigenicity of the intended therapeutic and adverse immune responses in subjects that receive such products.

### SUMMARY

Accordingly, needs have arisen for improved enzymes and processes for producing RNA products in vitro having less (e.g., little or no) duplex RNA. The present disclosure relates to systems, apparatus, compositions, enzymes and/or methods for producing RNA with little or no duplex RNA. For example, the present disclosure provides, according to some embodiments RNA polymerases for producing RNA with little or no duplex RNA including H6 RNA polymerase (e.g., SEQ ID NO: 21) and variants thereof (e.g., polymerases of Table 1). An H6 RNA polymerase may have an amino acid sequence at least 95% identical to SEQ ID NO: 21. A variant H6 RNA polymerase, in some embodiments, may have (a) an amino acid sequence at least 98% identical to any of SEQ ID NOS: 1-20, and (b) at least one substitution relative to SEQ ID NO: 21. According to some embodiments, a variant H6 RNA polymerase may have (a) an amino acid sequence at least 95% identical to any of SEQ ID NOS: 1-20, and (b) at least one substitution (e.g., one, two, three, four, five, six, seven, ten, or more substitutions) relative to SEQ ID NO: 21 at a position selected from positions corresponding to positions 106, 108, 134, 215, 232, 283, 327, 329, 378, 402, 419, 454, 459, 480, 481, 484, 490, 516, 519, 538, 715, 717, 756, 759, 798, 803, and 848 of SEQ ID NO: 21. A variant H6 RNA polymerase may have (a) an amino acid sequence at least 95% identical to any of SEQ ID NOS: 1-20, and (b) at least one substitution (e.g., one, two, three, four, five, six, or seven substitutions) relative to SEQ ID NO: 21 at a position selected from positions corresponding to positions 215, 459, 538, 759, 798, 803, and 848 of SEQ ID NO: 21 in some embodiments. Any of these variant H6 RNA polymerases may have a second substitution at a position (i) other than the position of the first substitution and (ii) corresponding to positions 215, 459, 538, 759, 798, 803, and 848 of SEQ ID NO: 21. And any of these variant H6 RNA polymerases may have a third substitution at a position (i) other than the position of the first and second substitutions and (ii) corresponding to positions 215, 459, 538, 759, 798, 803, and 848 of SEQ ID NO: 21. According to some embodiments, a variant H6 RNA polymerase may have (a) an amino acid sequence at least 95% identical to any of SEQ ID NOS: 1-20, and (b) substitutions relative to SEQ ID NO: 21 at positions corresponding to positions 215, 459, 538, 759, 798, 803, and 848 of SEQ ID NO: 21.

The present disclosure also relates to fusion proteins, which may, for example, comprise a peptide or protein of interest and an RNA polymerase for producing RNA with little or no duplex RNA including H6 RNA polymerase (e.g., SEQ ID NO: 21) and variants thereof (e.g., polymerases of Table 1). For example, a fusion may comprise, in an N-terminal to C-terminal direction, (I) a purification tag or a sorting signal peptide, and (II) a wild type or variant H6 RNA polymerase (e.g., according to Table 1) operably linked to (I). A fusion may comprise, in an N-terminal to C-terminal direction, (III) a wild type or variant H6 RNA polymerase (e.g., according to Table 1) and (IV) a purification tag or a sorting signal peptide operably linked to (III).

Compositions are also provided, which may comprise, for example, any of the disclosed H6 RNA polymerases or H6 polymerase fusions. Compositions may optionally include one or more of a buffering agent (e.g., HEPES, MES, MOPS, TAPS, tricine, Tris, ACES, ADA, BES, Bicine, CAPS, carbonic acid/bicarbonic acid, CHES, citric acid, DIPSO, EPPS, histidine, MOPSO, phosphoric acid, PIPES, POPSO, TAPS, TAPSO, triethanolamine), an excipient, a salt, a protein, a stabilizer, a detergent, a polyanion (e.g., spermidine, spermine, putrescine, polyethylenimine, 1,4,7-triazacyclononane, cyclen, ethylenediamine, 1,3,5,-triazinane), a polynucleotide (e.g., a template encoding an RNA of interest), a cell, a biological fluid or secretion, an aptamer, a pH indicator, a crowding agent, a sugar, a starch, cellulose, a glass-forming agent, a lipid, an oil, aqueous media, and/or a support. In some embodiments, a template may comprise an H6 RNA polymerase promoter (e.g., a promoter having a nucleotide sequence ≥90% or 100% identical to the sequence of any of SEQ ID NOS: 23-27).

The present disclosure further relates to methods for making an RNA of interest. An RNA of interest may be, for example, a messenger RNA (mRNA), ribosomal RNA (rRNA), transfer RNA (tRNA), small RNA (sRNA), microRNA (miRNA), long noncoding RNA (lncRNA), circular RNA (circRNA), aptamer RNA, antisense RNA, silencing RNA (siRNA), guide RNA (gRNA), or any combination thereof. A method of making an RNA of interest may include, for example, contacting (a) an H6 RNA polymerase (e.g., a wild type polymerase or a variant according to Table 1, an H6 RNA polymerase having at least 95%, at least 97%, at least 98%, or at least 99% identity to SEQ ID NO: 21), (b) a template encoding the RNA of interest, (c) optionally, one or more NTPs, (d) optionally, one or more modified NTPs, and (e) optionally, a buffer, to produce the RNA of interest, wherein the contacting is at a temperature in a range of 45° C.-65° C. and/or for a time in the range of seconds to hours and/or the RNA of interest is optionally a therapeutic RNA. In some embodiments, the RNA of interest may comprise a capped RNA and contacting further comprising contacting the polymerase, the template, the optional components, if present, and a capping enzyme to produce the capped RNA. A method may further include, according to some embodiments, contacting the RNA of interest with one or more pharmaceutically acceptable additives. A template, according to some embodiments, may further comprise an H6 RNA polymerase promoter (e.g., a promoter having a nucleotide sequence ≥90% or 100% identical to the sequence of any of SEQ ID NOS: 23-27). An RNA of interest may comprise a capped RNA and the contacting may further comprise contacting the polymerase, the template, the optional components, if present, and a capping enzyme to produce the capped RNA. Methods may further incldue contacting the RNA of interest with one or more more pharmaceutically acceptable additives.

The present disclosure further relates, in some embodiments, to kits for making an RNA of interest. A kit may comprise, for example, an H6 RNA polymerase (e.g., a wild type polymerase or a variant according to Table 1, an H6 RNA polymerase having at least 95%, at least 97%, at least 98%, or at least 99% identity to SEQ ID NO: 21) and one or more NTPs and/or one more more modified NTPs and/or a capping enzyme and/or a cap analog. A kit may comprise, for example, an H6 RNA polymerase and one or more NTPs. A kit may further comprise one or more modified NTPs. A kit may further comprise one or more capping enzymes. In some embodiments, a kit may comprise a reaction buffer (e.g., an in vitro transcription reaction buffer) and/or a polyamine. Example buffering agents include HEPES, MES, MOPS, TAPS, tricine, Tris, ACES, ADA, BES, Bicine, CAPS, carbonic acid/bicarbonic acid, CHES, citric acid, DIPSO, EPPS, histidine, MOPSO, phosphoric acid, PIPES, POPSO, TAPS, TAPSO, and triethanolamine. Example polyamines include spermidine, spermine, putrescine, polyethylenimine, 1,4,7-triazacyclononane, cyclen, ethylenediamine, and 1,3,5,-triazinane.

### BRIEF DESCRIPTION OF THE FIGURES

FIG. 1A shows an example embodiment of a promoter sequence (SEQ ID NO: 24) of H6 RNA polymerase in a comparison with other commonly used RNA polymerases. The consensus sequences are highlighted with gray (partially conserved) and black (fully conserved) backgrounds. The promoters of T7 (SEQ ID NO: 29) and SP6 polymerases (SEQ ID NO: 31) share 65% nucleotide identity with the H6 promoter while the proteins share 38% residue identity. H6 is specific for its own promoter sequences, displaying no cross activation of T7 promoters or other promoters tested. FIG. 1B shows an example SDS PAGE analysis of purified T7 RNA polymerase, H6 RNA polymerase, and Hi-H6 RNA polymerase.

FIG. 2 shows the melting temperatures (Tm) of T7 RNA polymerase and example embodiments of H6 RNA polymerase and Hi-H6 RNA polymerases, in each case, measured using nano-differential scanning fluorimetry. As shown, the observed Tm for H6 RNA polymerase is 6.5° C. higher than the observed Tm for T7 RNA polymerase and the observed Tm for Hi-H6 RNA polymerase is 8° C. higher than the observed Tm for H6 RNA polymerase.

FIG. 3A shows an example denaturing urea PAGE analysis demonstrating the integrity of a 50-mer transcript in vitro transcribed with T7 and H6 RNA polymerases at temperatures from 37° C. to 65° C. FIG. 3B shows example yields of the 50-mer transcripts in vitro transcribed with T7 and H6 RNA polymerases at temperatures where the RNA polymerases are active. T7 RNA polymerase performed well at 40° C. and was active at 45° C. while H6 RNA polymerase performed well at 45° C. and was active at 55° C.

FIG. 4A shows example agarose gel electrophoresis demonstrating the integrity of a 1-kb transcript RNA1 in vitro transcribed with T7 and H6 RNA polymerases at temperatures from 37° C. to 65° C. RNA1 represents artificial RNA sequences that have been permutated to include every four-base combination. FIG. 4B shows example yields of RNA1 in vitro transcribed with T7 and H6 RNA polymerases at temperatures where the RNA polymerases are active. Consistent with results shown in FIGS. 3A and 3B, T7 RNA polymerase performed well at 40° C. and was active at 45° C. while H6 RNA polymerase performed well at 45° C. and was active at 55° C.

FIG. 5A shows an example agarose gel electrophoresis analysis demonstrating the integrity of a 1.7-kb Cypridina luciferase (CLuc) mRNA in vitro transcribed with T7 and H6 RNA polymerases at temperatures from 37° C. to 65° C. FIG. 5B shows example yield of the CLuc mRNA in vitro transcribed with T7 and H6 RNA polymerases at temperatures where the RNA polymerases are active. Consistent with results shown in FIGS. 3A, 3B, 4A, and 4B, T7 RNA polymerase performed well at 40° C. and was active at 45° C., but here H6 RNA polymerase performed well at 45° C. and was active at 50° C. The shift in activity at higher temperatures may indicate that transcriptional activity at higher temperatures is a function of the enzyme used and the sequence to be transcribed.

FIGS. 6A-6N show example mass spectra demonstrating the non-DNA-templated additions to a 30-nt oligonucleotide transcribed in vitro with T7 RNA polymerase and H6 RNA polymerase at its active temperatures on a C-depleted DNA template. Run-off product (R) having up to two additional nucleotides and extended product (E) having three or more nucleotides are labeled. FIGS. 6A-6F show mass spectra of IVT reactions performed with T7 RNA polymerase. FIGS. 6G-6N show mass spectra of IVT reactions performed with H6 RNA polymerase. FIGS. 6A, 6C, 6E, 6G, 6I, 6K, and 6M show mass spectra of IVT reactions performed with ATP, GTP and UTP, but without CTP. FIGS. 6B, 6D, 6F, 6H, 6J, 6L, and 6N show mass spectra of IVT reactions performed with all four NTPs, namely ATP, CTP, GTP and UTP. FIGS. 6A-6B and 6G-6H show mass spectra of IVT reactions performed at 37° C. FIGS. 6C-6D and 6I-6J show mass spectra of IVT reactions performed at 40° C. FIGS. 6E-6F and 6K-6L show mass spectra of IVT reactions performed at 45° C. FIGS. 6M-6N show mass spectra of IVT reactions performed at 50° C. Reactions that did not include CTP were not expected to produce any extended (E) products because the respective polymerases should stall at any G in the template strand. On the other hand, reactions that did include CTP afford the respective polymerases the opportunity to extend the nascent RNA strand from its 3′ end (beyond the template strand). If extended sufficiently, the nascent strand may loop back on itself to form dsRNA which may include a hairpin. dsRNA formation at higher temperatures was reduced for both RNAPs.

FIG. 7A and FIG. 7B show example capillary electrophoresis (CE) traces of 5′ FAM-labeled 24-nt ribooligonucleotide incubated with T7 RNA polymerase (FIG. 7A) or H6 RNA polymerase (FIG. 7B) under IVT conditions at temperatures from 37° C. to 65° C. These IVT reactions had no DNA template such that the only available substrate for the respective RNA polymerases was the FAM-labeled RNA. Substrate (S) and extended oligonucleotide (E) peaks are labeled. H6 RNA polymerase is active in IVT reactions at 50° C. and 55° C. but, according to these results, does not produce dsRNA at this temperature.

FIG. 8A shows example agarose gel electrophoresis results demonstrating the integrity of a 1.7-kb Cypridina luciferase (CLuc) mRNA in vitro transcribed with T7 and H6 RNA polymerases at 37° C. and 45° C., respectively, after a 10-minute pre-incubation at temperatures from 37° C. to 65° C. FIG. 8B shows example yield of CLuc mRNA in vitro transcribed achieved with T7 and H6 RNA polymerases at 37° C. and 45° C., respectively, after a 10-minute pre-incubation at temperatures from 37° C. to 65° C. (indicated above each lane). FIG. 8C shows example agarose gel electrophoresis results demonstrating the integrity of a 1.7-kb Cypridina luciferase (CLuc) mRNA in vitro transcribed with H6 and H6 (N538H; SEQ ID NO: 18) RNA polymerases at 45° C. after a 10-minute pre-incubation at temperatures from 37° C. to 65° C. (indicated above each lane). FIG. 8D shows an example of normalized fluorescent data illustrating the coupled in vitro transcription/translation yield of a fast-maturing fluorescent protein (H6::vGFP) driven by H6 RNA polymerase or H6 (N538H) RNA polymerase after a 30-minute pre-incubation of the polymerases at temperatures from 45° C. to 65° C. The thermally challenged proteins and H6::vGFP template were mixed with NEBExpress® S30 lysate lacking RNA polymerase and fluorescent protein production was determined 3-hours after incubation at 37° C.

FIG. 9A shows an example agarose gel electrophoresis demonstrating the integrity of 1.7-kb CLuc mRNA in vitro transcript with the incorporation of either uridine (U), pseudouridine (Ψ), or N1-methyl-pseudouridine (m1Ψ) by H6 RNA polymerase at 50° C. FIG. 9B shows the yield of the example IVT reactions of FIG. 9A. Taken together, these data show that at least H6 RNA polymerase incorporated uridine analogs.

FIG. 10A shows example results of denaturing urea PAGE demonstrating the integrity of a 50-mer transcript transcribed in vitro with Hi-H6 RNA polymerase at temperatures from 37° C. to 65° C. FIG. 10B shows the yield of the example IVT reactions of FIG. 10A.

FIG. 11A shows agarose gel electrophoresis demonstrating the integrity of RNA1 in vitro transcribed with Hi-H6 RNA polymerases at temperatures from 37° C. to 65° C. RNA1 represents artificial RNA sequences that have been permutated to include every four-base combination. FIG. 11B shows the yield of the example IVT reactions of FIG. 11A.

FIG. 12A shows agarose gel electrophoresis demonstrating the integrity of a 1.7-kb CLuc mRNA in vitro transcribed with Hi-H6 RNA polymerase at temperatures from 37° C. to 65° C. FIG. 12B shows the yield of the example IVT reactions of FIG. 12A.

FIGS. 13A-13L show example mass spectra demonstrating the non-DNA-templated additions to a 30-nt oligonucleotide transcribed in vitro with Hi-H6 RNA polymerase at its active temperatures on a C-depleted DNA template. Run-off product (R) having up to two additional nucleotides and extended product (E) having three or more nucleotides are labeled. FIGS. 13A, 13C, 13E, 13G, 13I, and 6K show mass spectra of IVT reactions performed with ATP, GTP and UTP, but without CTP. FIGS. 6B, 6D, 6F, 6H, 6J, and 13L show mass spectra of IVT reactions performed with all four NTPs, namely ATP, CTP, GTP and UTP. FIGS. 13A-13B show mass spectra of IVT reactions performed at 37° C. FIGS. 13C-13D show mass spectra of IVT reactions performed at 40° C. FIGS. 13E-13F show mass spectra of IVT reactions performed at 45° C. FIGS. 13G-13H show mass spectra of IVT reactions performed at 50° C. FIGS. 13I-13J show mass spectra of IVT reactions performed at 55° C. FIGS. 13K-13L show mass spectra of IVT reactions performed at 60° C. As described in connection with FIGS. 6A-6N, reactions that did not include CTP were not expected to produce any extended (E) products because the respective polymerases should stall at any G in the template strand, while reactions that did include CTP afford the respective polymerases the opportunity to extend the nascent RNA strand from its 3′ end (beyond the template strand). If extended sufficiently, the nascent strand may loop back on itself to form dsRNA which may include a hairpin.

FIG. 14 shows example capillary electrophoresis (CE) traces of 5′ FAM-labeled 24-nt ribooligonucleotide incubated with Hi-H6 RNA polymerase in IVT conditions at temperatures from 37° C. to 65° C. These IVT reactions had no DNA template such that the only available substrate for the respective RNA polymerases was the FAM-labeled RNA. Substrate (S) and extended oligonucleotide (E) peaks are labeled. Hi-H6 RNA polymerase is active in IVT reactions at 60° C. but, according to these results, does not produce dsRNA at this temperature.

FIG. 15A shows an example agarose gel electrophoresis analysis demonstrating the integrity of a 1.7-kb CLuc mRNA in vitro transcribed with Hi-H6 RNA polymerase at 50° C. after a 10-minute pre-incubation at temperatures from 37° C. to 65° C. (indicated above each lane). FIG. 15B shows the yield of the example IVT reactions of FIG. 15A.

FIG. 16A shows an example agarose gel electrophoresis analysis demonstrating the integrity of 1.7-kb CLuc mRNA in vitro transcript with the incorporation of uridine (U), pseudouridine (Ψ), or N1-methyl-pseudouridine (m1Ψ) by Hi-H6 RNA polymerase at 50° C. FIG. 16B shows the yield of the example IVT reactions of FIG. 16A.

### BRIEF DESCRIPTION OF THE SEQUENCES

Some embodiments of this disclosure relate to the following provided sequences of example polynucleotides and/or example polypeptides.

SEQ ID NO: 1 is an example variant H6 RNA polymerase, namely Hi H6 RNA polymerase having an M215I substitution (numbering according to SEQ ID NO: 21).

SEQ ID NO: 2 is an example variant H6 RNA polymerase, namely Hi H6 RNA polymerase having an S459N substitution (numbering according to SEQ ID NO: 21).

SEQ ID NO: 3 is an example variant H6 RNA polymerase, namely Hi H6 RNA polymerase having an N538H substitution (numbering according to SEQ ID NO: 21).

SEQ ID NO: 4 is an example variant H6 RNA polymerase, namely Hi H6 RNA polymerase having a W759F substitution (numbering according to SEQ ID NO: 21).

SEQ ID NO: 5 is an example variant H6 RNA polymerase, namely Hi H6 RNA polymerase having a W798D substitution (numbering according to SEQ ID NO: 21).

SEQ ID NO: 6 is an example variant H6 RNA polymerase, namely Hi H6 RNA polymerase having a F803I substitution (numbering according to SEQ ID NO: 21) (numbering according to SEQ ID NO: 21).

SEQ ID NO: 7 is an example variant H6 RNA polymerase, namely Hi H6 RNA polymerase having an H848K substitution (numbering according to SEQ ID NO: 21).

SEQ ID NO: 8 is an example variant H6 RNA polymerase, namely Hi H6 RNA polymerase having M215I, S459N, N538H, W759F, W798D, F803I, and H848K substitutions (numbering according to SEQ ID NO: 21).

SEQ ID NO: 9 is an example variant H6 RNA polymerase, namely Hi H6 RNA polymerase having A106L, H108D, L134R, M215I, T232V, N283E, Q327D, T329I, T378K, V402Q, A419P, A454K, S459N, A480D, A481D, A484D, Y490L, R516E, F519S, N538H, M715W, E717P, S756P, W759F, W798D, F803I, and H848K substitutions (numbering according to SEQ ID NO: 21).

SEQ ID NO: 10 is an example variant H6 RNA polymerase, namely Hi H6 RNA polymerase having A106L, H108D, L134R, M215I, T232V, N283E, Q327D, T329I, T378K, V402S, A419P, A454K, S459N, A480D, A481D, A484D, Y490L, R516E, F519S, N538H, M715W, E717P, S756P, W759F, W798D, F803I, and H848K substitutions (numbering according to SEQ ID NO: 21).

SEQ ID NO: 11 is an example variant H6 RNA polymerase, namely Hi H6 RNA polymerase having an M215I substitution (numbering according to SEQ ID NO: 21), wherein the sequence includes (N-terminus to C-terminus) eight amino acids that enhance protein expression, followed by a polyhistidine tag (amino acids 9-14), a two amino acid linker (amino acids 15-16), and the polymerase sequence (amino acids 17-874).

SEQ ID NO: 12 is an example variant H6 RNA polymerase, namely Hi H6 RNA polymerase having an S459N substitution (numbering according to SEQ ID NO: 21), wherein the sequence includes (N-terminus to C-terminus) eight amino acids that enhance protein expression, followed by a polyhistidine tag (amino acids 9-14), a two amino acid linker (amino acids 15-16), and the polymerase sequence (amino acids 17-874).

SEQ ID NO: 13 is an example variant H6 RNA polymerase, namely Hi H6 RNA polymerase having an N538H substitution (numbering according to SEQ ID NO: 21), wherein the sequence includes (N-terminus to C-terminus) eight amino acids that enhance protein expression, followed by a polyhistidine tag (amino acids 9-14), a two amino acid linker (amino acids 15-16), and the polymerase sequence (amino acids 17-874).

SEQ ID NO: 14 is an example variant H6 RNA polymerase, namely Hi H6 RNA polymerase having a W759F substitution (numbering according to SEQ ID NO: 21), wherein the sequence includes (N-terminus to C-terminus) eight amino acids that enhance protein expression, followed by a polyhistidine tag (amino acids 9-14), a two amino acid linker (amino acids 15-16), and the polymerase sequence (amino acids 17-874).

SEQ ID NO: 15 is an example variant H6 RNA polymerase, namely Hi H6 RNA polymerase having a W798D substitution (numbering according to SEQ ID NO: 21), wherein the sequence includes (N-terminus to C-terminus) eight amino acids that enhance protein expression, followed by a polyhistidine tag (amino acids 9-14), a two amino acid linker (amino acids 15-16), and the polymerase sequence (amino acids 17-874).

SEQ ID NO: 16 is an example variant H6 RNA polymerase, namely Hi H6 RNA polymerase having an F803I substitution (numbering according to SEQ ID NO: 21), wherein the sequence includes (N-terminus to C-terminus) eight amino acids that enhance protein expression, followed by a polyhistidine tag (amino acids 9-14), a two amino acid linker (amino acids 15-16), and the polymerase sequence (amino acids 17-874).

SEQ ID NO: 17 is an example variant H6 RNA polymerase, namely Hi H6 RNA polymerase having an H848K substitution (numbering according to SEQ ID NO: 21), wherein the sequence includes (N-terminus to C-terminus) eight amino acids that enhance protein expression, followed by a polyhistidine tag (amino acids 9-14), a two amino acid linker (amino acids 15-16), and the polymerase sequence (amino acids 17-874).

SEQ ID NO: 18 is an example variant H6 RNA polymerase, namely Hi H6 RNA polymerase having M215I, S459N, N538H, W759F, W798D, F803I, and H848K substitutions (numbering according to SEQ ID NO: 21), wherein the sequence includes (N-terminus to C-terminus) eight amino acids that enhance protein expression, followed by a polyhistidine tag (amino acids 9-14), a two amino acid linker (amino acids 15-16), and the polymerase sequence (amino acids 17-874).

SEQ ID NO: 19 is an example variant H6 RNA polymerase, namely Hi H6 RNA polymerase having A106L, H108D, L134R, M215I, T232V, N283E, Q327D, T329I, T378K, V402Q, A419P, A454K, S459N, A480D, A481D, A484D, Y490L, R516E, F519S, N538H, M715W, E717P, S756P, W759F, W798D, F803I, and H848K substitutions (numbering according to SEQ ID NO: 21), wherein the sequence includes (N-terminus to C-terminus) eight amino acids that enhance protein expression, followed by a polyhistidine tag (amino acids 9-14), a two amino acid linker (amino acids 15-16), and the polymerase sequence (amino acids 17-874).

SEQ ID NO: 20 is an example variant H6 RNA polymerase, namely Hi H6 RNA polymerase having A106L, H108D, L134R, M215I, T232V, N283E, Q327D, T329I, T378K, V402S, A419P, A454K, S459N, A480D, A481D, A484D, Y490L, R516E, F519S, N538H, M715W, E717P, S756P, W759F, W798D, F803I, and H848K substitutions (numbering according to SEQ ID NO: 21), wherein the sequence includes (N-terminus to C-terminus) eight amino acids that enhance protein expression, followed by a polyhistidine tag (amino acids 9-14), a two amino acid linker (amino acids 15-16), and the polymerase sequence (amino acids 17-874).

SEQ ID NO: 21 is an example wild type H6 RNA polymerase of Autographiviridae sp. isolate UViG_3300025572_000021. One or more of positions 106, 108, 134, 215, 232, 283, 327, 329, 378, 402, 419, 454, 459, 480, 481, 484, 490, 516, 519, 538, 715, 717, 756, 759, 798, 803, and 848 may be substituted as disclosed herein.

SEQ ID NO: 22 is an example variant H6 RNA polymerase comprising a wild type H6 RNA polymerase sequence (SEQ ID NO: 21) and an N-terminal leader, wherein the sequence includes (N-terminus to C-terminus) seven amino acids that enhance protein expression, followed by a polyhistidine tag (amino acids 8-13), a two amino acid linker (amino acids 14-15), and the polymerase sequence (amino acids 16-873).

SEQ ID NO: 23 is an example promoter for an H6 RNA polymerase (e.g., a wild type H6 RNA polymerase or a variant H6 RNA polymerase).

SEQ ID NO: 24 is an example promoter for an H6 RNA polymerase (e.g., a wild type H6 RNA polymerase or a variant H6 RNA polymerase).

SEQ ID NO: 25 is an example promoter for an H6 RNA polymerase (e.g., a wild type H6 RNA polymerase or a variant H6 RNA polymerase).

SEQ ID NO: 26 is an example promoter for an H6 RNA polymerase (e.g., a wild type H6 RNA polymerase or a variant H6 RNA polymerase).

SEQ ID NO: 27 is an example promoter for an H6 RNA polymerase (e.g., a wild type H6 RNA polymerase or a variant H6 RNA polymerase).

SEQ ID NO: 28 is an example T7 RNA polymerase.

SEQ ID NO: 29 is an example promoter for T7 RNA polymerase.

SEQ ID NO: 30 is an example SP6 RNA polymerase.

SEQ ID NO: 31 is an example promoter for SP6 RNA polymerase.

SEQ ID NO: 32 is an example promoter for T3 RNA polymerase.

SEQ ID NO: 33 is an example 23-nt 5′-FAM labeled ribooligonucleotide.

SEQ ID NO: 34 is an example 30-nt oligonucleotide.

SEQ ID NO: 35 is an example 50-nt oligonucleotide.

SEQ ID NO: 36 is an example IVT transcript (“RNA1”) and includes every combination of 4 bases to reveal any sequence bias an RNA polymerase may have.

SEQ ID NO: 37 is an example IVT transcript (“CLuc”).

SEQ ID NO: 38 is an example sequence encoding an H6 RNA polymerase (“pJAB199”) [pET29c 6×His-H6 RNAP (wild-type, aa)].

SEQ ID NO: 39 is an example sequence encoding an H6 RNA polymerase GFP fusion (“pJAB201”) [pUC19 P_{H6}::VGFP].

SEQ ID NO: 40 is an example variant H6 RNA polymerase, wherein the sequence includes (N-terminus to C-terminus) an N-terminal methionine for expression, followed by a polyhistidine tag (amino acids 2-7), a two amino acid linker (amino acids 8-9), and the wild type polymerase sequence (amino acids 10-858). One or more of positions 115, 117, 143, 224, 241, 292, 336, 338, 387, 411, 428, 463, 468, 489, 490, 493, 499, 525, 528, 547, 724, 726, 765, 768, 807, 814, and 857 may be substituted as disclosed herein.

### DETAILED DESCRIPTION

The present disclosure relates, in some embodiments, to RNA polymerases and methods, systems, and workflows including RNA polymerases. According to some embodiments, an RNA polymerase may include an H6 RNA polymerase (e.g., SEQ ID NO: 21) and/or variants thereof (e.g., variants having ≥90%, ≥95%, ≥98% to any of SEQ ID NOS: 1-20 and/or 22).

### General Considerations

Aspects of the present disclosure can be understood in light of the provided descriptions, figures, sequences, embodiments, section headings, and examples, none of which should be construed as limiting the entire scope of the present disclosure in any way. Accordingly, the innovations set forth herein should be construed in view of the full breadth and spirit of the disclosure.

Each of the individual embodiments described and illustrated herein has discrete components and features which can be readily separated from or combined with the components and/or features of any of the other several embodiments without departing from the scope or spirit of the present teachings. Lists of example species within a particular genus may vary in length at different places throughout the disclosure. Species lists shortened for convenience shall not be construed to exclude example species listed elsewhere in the specification. Any recited method can be carried out in the order of events recited or in any other order which is logically possible. Unless otherwise expressly stated to be required herein, each component, feature, and method step disclosed herein is optional and the disclosure contemplates embodiments in which each optional element may be expressly excluded. As such, this statement is intended to serve as antecedent basis for use of such exclusive terminology as “solely,” “only” and the like in connection with the recitation of claim elements or use of a “negative” limitation. It is further intended to serve as antecedent basis for use of such elective terminology as “optionally” and the like in connection with the recitation of one or more claim elements.

Unless otherwise defined, all technical and scientific terms used herein have the same meaning as commonly understood by one of ordinary skill in the art to which this disclosure belongs. Still, certain terms are defined herein with respect to embodiments of the disclosure and for the sake of clarity and ease of reference.

Sources of commonly understood terms and symbols may include: standard treatises and texts such as Kornberg and Baker, DNA Replication, Second Edition (W. H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); Singleton, et al., Dictionary of Microbiology and Molecular biology, 2d ed., John Wiley and Sons, New York (1994), and Hale & Markham, the Harper Collins Dictionary of Biology, Harper Perennial, N.Y. (1991) and the like.

As used herein and in the appended claims, the singular forms “a” and “an” include plural referents unless the context clearly dictates otherwise. For example, the term “a protein” refers to one or more proteins, i.e., a single protein and multiple proteins.

Numeric ranges are inclusive of the numbers defining the range. All numbers should be understood to encompass the midpoint of the integer above and below the integer i.e., the number 2 encompasses 1.5-2.5. The number 2.5 encompasses 2.45-2.55 etc. When sample numerical values are provided, each alone may represent an intermediate value in a range of values and together may represent the extremes of a range unless specified. Percent ranges with only one end point (e.g., ≥90% or ≤10%) optionally include a second endpoint at the maximum or minimum percentage (e.g., ≥90% includes a range of 90%-100% and ≤10% includes a range of 0%-10%). Ranges (including percent ranges) with only one end point (e.g., ≥90 or ≤10) optionally include a second endpoint 10% higher or 10% lower than the provided endpoint (e.g., ≥90 includes a range of 90-99 and ≤10 includes a range of 1-10).

In the context of the present disclosure, “buffer” and “buffering agent” refer to a chemical entity or composition that itself resists and, when present in a solution, allows such solution to resist changes in pH when such solution is contacted with a chemical entity or composition having a higher or lower pH (e.g., an acid or alkali). Examples of suitable non-naturally occurring buffering agents that may be used in disclosed compositions, kits, and methods include HEPES, MES, MOPS, TAPS, tricine, and Tris. Additional examples of suitable buffering agents that may be used in disclosed compositions, kits, and methods include ACES, ADA, BES, Bicine, CAPS, carbonic acid/bicarbonic acid, CHES, citric acid, DIPSO, EPPS, histidine, MOPSO, phosphoric acid, PIPES, POPSO, TAPS, TAPSO, and triethanolamine.

In the context of the present disclosure, “container” refers to a human-made container. A container may comprise one or more walls (e.g., defining an interior volume) and optionally one or more openings. Containers comprising one or more openings may further comprise one or more closures (e.g., a removable closures) for some or all such openings. A closure optionally may comprise an aperture or a septum, for example, to provide fluid communication with a volume of the container and a connected or inserted tube or syringe. Examples of containers include boxes, cartons, bottles, tubes (e.g., test tubes, microcentrifuge tubes), plates (e.g., 96-well, 384-well plates), vials, pipette tips, and ampules. Containers and/or closures may comprise any desired material including paper, plastics, glass, silicone, composites, metals, alloys, or combinations thereof. Containers and/or closures may comprise materials that are compostable, recyclable, and/or sustainable.

In the context of the present disclosure and with respect to an amino acid residue or a nucleotide base position, “corresponding to” refers to positions that lie across from one another when sequences are aligned, e.g., by the BLAST algorithm. An amino acid position in a functional or structural motif in one polymerase may correspond to a position within a functionally equivalent functional or structural motif in another polymerase.

In the context of the present disclosure, “expression system” refers to systems for producing a protein from a polynucleotide template comprising components to produce the protein according to an RNA template (e.g., enzymes, amino acids, an energy source), (optionally) components to produce the RNA template according to another RNA template or a DNA template (e.g., enzymes, nucleotides, an energy source). An expression system may comprise a bacterial (e.g., Escherichia coli) or yeast (e.g., Kluyveromyces lactis or Pichia pastoris) expression system in which the protein is encoded by an RNA or DNA template within an expression cassette, a plasmid or other expression vector. An expression system may comprise a viral expression system in which the protein is encoded by an RNA or DNA template (e.g., in an expression cassette) within a viral genome or viral expression vector. Examples of cell-free expression systems may include or comprise cell extracts of Escherichia coli S30, rabbit reticulocytes or wheat germ, PUREEXPRESS® (New England Biolabs, Ipswich, MA), an insect cell extract system (e.g., Promega #L1101), or HeLa cell lysate-based protein expression systems (e.g., Thermo Fisher Scientific #88882). An expression cassette may comprise, in some embodiments, an expression control sequence (e.g., promoter), a coding sequence encoding the gene product (e.g., protein) of interest (e.g., a vaccinia capping enzyme fusion), and/or one or more termination sequences (e.g., terminators). An expression control sequence (e.g., promoter) may comprise any promoter operative in a desired expression system, including, for example, a GAP promoter, an AOX1 promoter, a LAC4 promoter, a P350 hybrid promoter, a T7 promoter, a T5 promoter, a Ptac promoter, a Ptrc promoter, ParaBAD promoter, a PrhaBAD promoter, a Tet promoter or a PhoA phosphate-starvation promoter.

In the context of the present disclosure, “fusion” refers to two or more polypeptides, subunits, or proteins covalently joined to one another (e.g., by a peptide bond). For example, a protein fusion may refer to a non-naturally occurring polypeptide comprising a protein of interest covalently joined to a second polypeptide. Examples of a second polypeptide include a reporter protein (e.g., a green fluorescent protein), a purification tag, and expression tag, a polynucleotide binding protein, an enzyme (e.g., a capping enzyme), a conjugation tag (e.g., a SNAP® tag), and a peptide linker. Unless otherwise disclosed, the protein of interest may be nearer to the N-terminal end or nearer to the C-terminal end than the second polypeptide to which it is joined. A fusion may comprise a non-naturally occurring combined polypeptide chain comprising two proteins or two protein domains joined directly to each other by a peptide bond or joined through a peptide linker. In some embodiments, a fusion may comprise an H6 RNA polymerase (e.g., a wt or variant H6 RNA polymerase) covalently joined to a second polypeptide. In some embodiments, a variant H6 RNA polymerase may include a fusion to an exogenous DNA binding domain. Examples are provided in Table 1 of U.S. Pat. No. 11,259,184.

In the context of the present disclosure, “immobilized” refers to covalent attachment of an enzyme to a solid support with or without a linker. Examples of solid supports include beads (e.g., magnetic, agarose, polystyrene, polyacrylamide, chitin). Beads may include one or more surface modifications (e.g., O⁶-benzyleguanine, polyethylene glycol) that facilitate covalent attachment and/or activity of an enzyme of interest. For example, a support may comprise a ligand and an enzyme may have a receptor for such ligand or an enzyme may comprise a ligand and a support may comprise a receptor for such ligand. Receptor-ligand binding may be covalent or non-covalent. Non-covalent attachment (e.g., avidin:biotin, chitin:CBP) may be useful in some embodiments, for example, where the level of dissociation of the binding partner is deemed tolerable. A linker may be disposed between a support and an enzyme. For example, linker disposed between a support and an enzyme may have a first covalent bond to the support and a second covalent bond to the enzyme. An immobilized enzyme comprising a ligand-receptor attachment may have a linker disposed between the support and the ligand-receptor attachment, a linker disposed between the enzyme and the ligand-receptor attachment, or both. An immobilized enzyme comprising a linker may also comprise an optional covalent bond directly between the enzyme and the support. A linker may be of any desired length and have any desired range of motion. A peptide linker may comprise one or more repeats (e.g., 1-10 repeats) of glycine-serine.

In the context of the present disclosure, “in vitro transcription” (IVT) refers to a cell-free reaction in which a DNA template is copied by a DNA-directed RNA polymerase (e.g., an H6 RNA polymerase) to produce a product that comprises one or more RNA molecules having a sequence copied from the template.

In the context of the present disclosure, “modified nucleotide” refers to nucleotides having a modification on the sugars (e.g., 2′-fluororibose, ribose, 2′-deoxyribose, arabinose, and hexose); and/or in the phosphate groups (e.g., phosphorothioates and 5′-N-phosphoramidite linkages); and/or in the nucleotide base (e.g., as described in U.S. Pat. No. 8,383,340; WO 2013/151666; U.S. Pat. No. 9,428,535 B2; US 2016/0032316). Examples of modified nucleotides include pseudouridine and N1-methyl-pseudouridine.

In the context of the present disclosure, “non-naturally occurring” refers to a molecule (e.g., a polynucleotide, polypeptide, carbohydrate, or lipid) or composition that does not exist in nature. Such a molecule or composition may differ from naturally occurring molecules or compositions in one or more respects. For example, a polymer (e.g., a polynucleotide, polypeptide, or carbohydrate) may differ in the kind and arrangement of the component parts (e.g., nucleotide sequence, amino acid sequence, or sugar molecules). A polymer may differ from a naturally occurring polymer with respect to the molecule(s) to which it is linked. For example, a “non-naturally occurring” polypeptide (e.g., protein) may differ from naturally occurring polypeptides in its secondary, tertiary, or quaternary structure, by having (or lacking) a chemical bond (e.g., a covalent bond including a peptide bond, a phosphate bond, a disulfide bond, an ester bond, and ether bond, and others) to a lipid, a carbohydrate, a second polypeptide (e.g., a fusion protein), or any other molecule. Similarly, a “non-naturally occurring” polynucleotide or nucleic acid may comprise (or lack) one or more other modifications (e.g., an added label or other moiety) to the 5′-end, the 3′ end, and/or between the 5′- and 3′-ends (e.g., methylation) of the nucleic acid. A “non-naturally occurring” molecule or composition may differ from naturally occurring compositions in one or more of the following respects: (a) having components that are not combined in nature, (b) having components in ratios and/or concentrations not found in nature, (c) lacking one or more components otherwise found in naturally occurring molecules or compositions (e.g., a cell-free composition, a chromosome-free composition, a histone-free composition, a polymerase-free composition, a cell membrane-free composition), (d) having a form not found in nature (e.g., dried, freeze dried, lyophilized, crystalline, aqueous, immobilized), and (e) having one or more additional components beyond those found in nature (e.g., a buffering agent, a detergent, a dye, a solvent or a preservative).

In the context of the present disclosure, “polymerase” refers to an enzyme that synthesizes a polynucleotide from NTPs with or without a template. Examples of enzymes include T3 RNA polymerase, T7 RNA polymerase, SP6 polymerase, H6 RNA polymerase, among others and variants thereof including thermostable variants (e.g., International PCT Publication No. WO2017123748 and U.S. Pat. Nos. 10,519,431 and 11,259,184).

With reference to an amino acid, “position” refers to the place such amino acid occupies in the primary sequence of a peptide or polypeptide numbered from its amino terminus to its carboxy terminus.

In the context of the present disclosure, “substitution” refers to an amino acid residue at a position in a comparator amino acid sequence that differs with respect to a corresponding position of a reference amino acid sequence, where the comparator and reference sequences are at least 60% identical to each other or at least 70% identical to each other or at least 80% identical to each other. A substitute amino acid residue at a position, in addition to differing from the corresponding position of a reference amino acid sequence, may differ from the amino acid at the corresponding position of all naturally-occurring sequences that are at least 60% identical to each other or at least 70% identical to each other or at least 80% identical to the reference sequence. A substitution may include replacing an amino acid with an amino acid having similar properties. For example, a polar amino acid may be replaced by another polar amino acid. A hydrophobic amino acid may be replaced by another hydrophobic amino acid. A charged amino acid (e.g., a positively charged amino acid or a negatively charged amino acid) may be replaced by another charged amino acid (e.g., a positively charged amino acid or a negatively charged amino acid). A substitution may include replacing an amino acid with an amino acid having different properties (e.g., different physical and/or chemical properties).

In the context of the present disclosure, “transcript” refers to a polynucleotide template for a polypeptide. A transcript may comprise RNA (e.g., ssRNA), a cap or cap analog, and/or a polyA tail. A transcript may be capable of translation in a cell (e.g., a bacterial cell and/or a yeast cell). For example, a transcript may be or comprise mRNA. A fusion transcript may comprise polynucleotide templates for two or more polypeptides in a single polynucleotide.

In the context of the present disclosure, “variant H6 RNA polymerase” refers to a non-naturally occurring enzyme that catalyzes template-dependent, 5′ to 3′ synthesis of RNA. A variant H6 RNA polymerase may have catalytic activity at and/or following exposure to temperatures in ranges X to Y, where X is any of 30° C., 35° C., 37° C., 40° C., 42° C., 45° C., 50° C. and Y is any of 45° C., 50° C., 55° C., 60° C., 65° C., 66° C., 68° C., 70° C. and X<Y. For example, a variant H6 RNA polymerase may have catalytic activity at and/or following exposure to temperatures in ranges 30° C.-70° C., 37° C.-65° C., 40° C.-65° C., or 45° C.-65° C. According to some embodiments, a variant H6 RNA polymerase has increased stability at temperatures of ≥37° C., ≥40° C., ≥42° C., ≥45° C., or ≥50° C. relative the T7 RNA polymerase of SEQ ID NO: 28 as a result of the one or more amino acid substitutions.

A variant H6 RNA polymerase may catalyze RNA synthesis more efficiently at any given temperature than a reference RNA polymerase, for example, wild type H6 RNA polymerase (SEQ ID NO: 21) or wild type T7 RNA polymerase. Increased efficiency of synthesizing a given RNA with a variant H6 RNA polymerase over a wildtype H6 RNA polymerase at 60° C. may be in a range X′ to Y′, wherein X′ is any of 5%, 10%, 25%, 50%, 75%, or 100% and Y′ is any of 10%, 25%, 50%, 75%, 100%, 250%, 500%, or 1000% (e.g., 5%-25%, 5%-100%, 10%-250%, 25%-1000% and X′<Y′).

Catalytic activity of a variant H6 RNA polymerase may persist across a range of salt concentrations, temperatures and/or pH. For example, a variant H6 RNA polymerase may display catalytic activity under a range of conditions and/or following removal from exposure to such conditions. A variant H6 RNA polymerase may have catalytic activity at and/or following exposure to a pH from X^{†} to Y^{†}, where X^{†} is any of pH 4, 4.5, 5, 5.5, 6, 6.5, 7, and Y^{†} is any of pH 6.5, 7, 7.5, 8, 8.5, 9, 9.5, 10, 10.5, 11 and X^{†}<^{†}.

A variant H6 RNA polymerase may comprise one or more amino acids in addition to a wild type H6 RNA polymerase (e.g., SEQ ID NO: 21). For example, a variant H6 RNA polymerase may comprise (e.g., at its amino terminal end or carboxy terminal end) 1-25 amino acids. Such additional amino acids may enable, facilitate and/or enhance translation, expression, cellular sorting, inactivation (e.g., by including a protease recognition and/or cleavage site), and/or purification. Such additional amino acids may consitute a linker, for example, to a support (e.g., a magnetic bead) or another protein.

A variant H6 RNA polymerase may have an amino acid sequence sharing any desired degree of sequence identity with wildtype H6 RNA polymerase (SEQ ID NO: 21) up to (but excluding) 100% identity. For example, a variant H6 RNA polymerase may have an amino sequence having at least 75%, at least 80%, at least 85%, at least 90%, at least 92%, at least 94%, at least 95%, at least 98%, or at least 99% identity to any of SEQ ID NOS: 1-20 (excluding 100% identity to SEQ ID NO: 21), optionally including a substitution at one or more (e.g., one, two, three, four, five, six, seven, ten, or all) of its positions that correspond to position 106, 108, 134, 215, 232, 283, 327, 329, 378, 402, 419, 454, 459, 480, 481, 484, 490, 516, 519, 538, 715, 717, 756, 759, 798, 803, and/or 848 of SEQ ID NO: 21. Example substitutions include A106L, H108D, L134R, M215I, T232V, N283E, Q327D, T329I, T378K, V402Q, V402S, A419P, A454K, S459N, A480D, A481D, A484D, Y490L, R516E, F519S, N538H, M715W, E717P, S756P, W759F, W798D, F803I, and H848K. Example variants are shown in Table 1 which sets forth the required degree of sequence identity to a reference wild type sequence and the position of required substitutions (numbering according to the reference sequence).

TABLE 1
Variant H6 RNA Polymerases

| | Reference | Substitution(s) | Example | Example |
|---|---|---|---|---|
| Sequence Identity | sequence | at | substitutions | SEQ ID NO |
| (a) ≥90%, ≥92%, ≥94%, ≥95%, ≥97%, ≥98%, | SEQ ID | 106 | A106L | 9, 10, 19, 20 |
| or ≥99%; and (b) <100% | NO: 21 | 108 | H108D | 9, 10, 19, 20 |
| | | 134 | L134R | 9, 10, 19, 20 |
| | | 215 | M215I | 1, 8-11, 18-20 |
| | | 232 | T232V | 9, 10, 19, 20 |
| | | 283 | N283E | 9, 10, 19, 20 |
| | | 327 | Q327D | 9, 10, 19, 20 |
| | | 329 | T329I | 9, 10, 19, 20 |
| | | 378 | T378K | 9, 10, 19, 20 |
| | | 402 | V402Q, V402S | 9, 10, 19, 20 |
| | | 419 | A419P | 9, 10, 19, 20 |
| | | 454 | A454K | 9, 10, 19, 20 |
| | | 459 | S459N | 2, 8-10, 12, 18-20 |
| | | 480 | A480D | 9, 10, 19, 20 |
| | | 481 | A481D | 9, 10, 19, 20 |
| | | 484 | A484D | 9, 10, 19, 20 |
| | | 490 | Y490L | 9, 10, 19, 20 |
| | | 516 | R516E | 9, 10, 19, 20 |
| | | 519 | F519S | 9, 10, 19, 20 |
| | | 538 | N538H | 3, 8-10, 13, 18-20 |
| | | 715 | M715W | 9, 10, 19, 20 |
| | | 717 | E717P | 9, 10, 19, 20 |
| | | 756 | S756P | 9, 10, 19, 20 |
| | | 759 | W759F | 4, 8-10, 14, 18-20 |
| | | 798 | W798D | 5, 8-10, 15, 18-20 |
| | | 803 | F803I | 6, 8-10, 16, 18-20 |
| | | 848 | H848K | 7-10, 17-20 |
| | | 215, 459, 538, | M215I, S459N, | 8-10, 18-20 |
| | | 759, 798, 803, | N538H, W759F, | |
| | | & 848 | W798D, F803I, | |
| | | | H848K | |
| | | 106, 108, 134, | A106L, H108D, | 9, 10, 19, 20 |
| | | 215, 232, 283, | L134R, M215I, | |
| | | 327, 329, 378, | T232V, N283E, | |
| | | 402, 419, 454, | Q327D, T329I, | |
| | | 459, 480, 481, | T378K, V402Q, | |
| | | 484, 490, 516, | V402S, A419P, | |
| | | 519, 538, 715, | A454K, S459N, | |
| | | 717, 756, 759, | A480D, A481D, | |
| | | 798, 803, & | A484D, Y490L, | |
| | | 848 | R516E, F519S, | |
| | | | N538H, M715W, | |
| | | | E717P, S756P, | |
| | | | W759F, W798D, | |
| | | | F803I, H848K | |

As shown in Table 1, for example, a variant H6 RNA polymerase may have an amino sequence having at least 90%, at least 92%, at least 94%, at least 95%, at least 97%, at least 98%, or at least 99% identity but less than 100% identity to SEQ ID NO: 21 and have a substitution at its position corresponding to position 538 of SEQ ID NO: 21 wherein an example substitution at this position is N538H and wherein example sequences for such variants include SEQ ID NOS: 3, 8-10, 13, and 18-20. A variant H6 RNA polymerase, as shown in TABLE 1 may have an amino sequence having at least 90%, at least 92%, at least 94%, at least 95%, at least 97%, at least 98%, or at least 99% identity but less than 100% identity to SEQ ID NO: 21 and have a substitutions at its position corresponding to position 215, 459, 538, 759, 798, 803, and 848 of SEQ ID NO: 21 wherein example substitutions at these positions include M215I, S459N, N538H, W759F, W798D, F803I, and H848K and wherein example sequences for such variants include SEQ ID NOS: 8-10 and 18-20.

All publications, patents, and patent applications mentioned in this specification are herein incorporated by reference to the same extent as if each individual publication, patent, or patent application was specifically and individually indicated to be incorporated by reference. Reagents referenced in this disclosure may be made using available materials and techniques, obtained from the indicated source, and/or obtained from New England Biolabs, Inc. (Ipswich, MA).

### Enzymes and Compositions

The present disclosure relates, in some embodiments, to H6 RNA polymerases (including variant H6 RNA polymerases) having one or more desirable properties including, for example, efficient and thermotolerant synthesis of RNA relative to, for example, wildtype T7 RNA protease.

The present disclosure relates, in some embodiments, to an immobilized enzyme comprising a support and an enzyme immobilized thereto. For example, an immobilized H6 RNA polymerase may comprise a H6 RNA polymerase (e.g., a wt or variant H6 RNA polymerase), a glycine-serine linker attached to the variant H6 RNA polymerase by a peptide bond, a protein tag (e.g., a SNAP-tag) attached to the linker by a peptide bond, O⁶-benzyleguanine bound to the protein tag (e.g., SNAP-Tag®); and beads (e.g., magnetic beads) having a surface modification comprising the O⁶-benzyleguanine. In some embodiments, a support of an immobilized H6 RNA polymerase (e.g., an immobilized variant H6 RNA polymerase) may comprise a magnetic bead. A magnetic bead may comprise, for example, one or more surface modifications. Surface modifications may include, for example, O⁶-benzyleguanine and/or PEG₇₅₀. In some embodiments, an immobilized enzyme may comprise a ligand (e.g., O⁶-benzyleguanine) and a receptor or tag (e.g., a SNAP-Tag®) capable of binding the ligand. For example, ligands may be disposed on a support and corresponding receptors may be disposed on (e.g., covalently attached to) an enzyme to be immobilized on the support. An immobilized enzyme may comprise, in some embodiments, an enzyme (e.g., variant H6 RNA polymerase), optionally, a first linker (e.g., a peptide linker) attached to the enzyme, a polypeptide tag (e.g., a SNAP-Tag®) attached to the first linker, if present, or the enzyme, a ligand corresponding to the polypeptide tag (e.g., O⁶-benzyleguanine) attached (e.g., covalently attached) to the tag, optionally, a second linker (e.g., polyethylene glycol) attached to the ligand, and a support (e.g., a magnetic bead) attached to the second linker if present or the ligand, the structure of which may be illustrated, in an N->C direction, as:

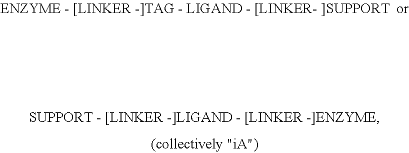

wherein dashes represent bonds (covalent or non-covalent) and brackets represent optional elements.

According to some embodiments, an H6 RNA polymerase composition may comprise an H6 RNA polymerase (e.g., a wildtype or variant H6 RNA polymerase) and, optionally, any of (including one or more of) a buffering agent (e.g., a storage buffer, a reaction buffer), an excipient, a salt (e.g., NaCl, MgCl₂, CaCl₂)), a protein (e.g., an internal control with or without a H6 RNA polymerase recognition sequence), a stabilizer, a detergent (for example, ionic, non-ionic, and/or zwitterionic detergents (e.g., octoxinol, polysorbate 20)), a polyanion (e.g., spermidine, spermine, putrescine), a polynucleotide (e.g., a template encoding an RNA of interest and optionally an H6 RNA polymerase promoter), a cell (e.g., intact, digested, or any cell-free extract), a biological fluid or secretion (e.g., mucus, pus, blood, urine, saliva), an aptamer, a pH indicator (e.g., azolitimin, bromocresol purple, bromothymol blue, methylene blue, cresol red, neutral red, naphtholphthalein, phenol red), a crowding agent, a sugar (e.g., a mono, di, tri, tetra, or higher saccharide), a starch, cellulose, a glass-forming agent (e.g., glycerol, raffinose, stachyose, or trehalose for lyophilization), a lipid, an oil, aqueous media, a support (e.g., a bead) and/or (non-naturally occurring) combinations thereof. Combinations may include for example, two or more of the listed components (e.g., a salt and a buffer) or a plurality of species of a single listed component (e.g., two different salts or two different sugars). In some embodiments, a composition may comprise 0.5-25 mM MgCl₂, e.g., 2 mM MgCl₂. Compositions may comprise one or more polyanions at any desired concentration (e.g., individually or total concentrations of 0.1-10 mM, 0.5-5 mM, 1 mM, 1.5 mM, 2 mM, 2.5 mM, 3 mM, 3.5 mM, 4 mM, 4.5 mM or 5 mM) and may be included to bind negatively charged molecules. Example polyanions include spermidine, spermine, putrescine, polyethylenimine, 1,4,7-triazacyclononane, cyclen, ethylenediamine, or 1,3,5,-triazinane. According to some embodiments, H6 RNA polymerase compositions may comprise (a) an H6 RNA polymerase (e.g., a wt or variant H6 RNA polymerase), (b) a buffer, and optionally (c) a polynucleotide (e.g., a template DNA) or a cellular extract or a cell-free preparation comprising a polynucleotide.

H6 RNA polymerases and variant H6 RNA polymerases described herein have RNA polymerase activity and, as such, have the capacity to catalyze the formation of RNA in the 5′→3′ direction using a DNA template. A DNA template may comprise a suitable promoter (e.g., a sequence having ≥85%, ≥90% or 100% identity to any of SEQ ID NOS: 23-27).

An H6 RNA polymerase composition may comprise, for example, a variant H6 RNA polymerase (e.g., having an amino acid sequence at least 85% identical to one or more of SEQ ID NO: 21 OR 22) and having at least one substitution, deletion, or insertion relative to wildtype H6 RNA polymerase. An H6 RNA polymerase composition may be free of one or more other catalytic activities. For example, an H6 RNA polymerase (e.g., a wt or variant H6 RNA polymerase) may be free of proteases (e.g., non-specific proteases or proteases having other cleavage recognition sites), free of nucleases (e.g., RNases and/or DNases), free of other polymerase activity, free of RNA and/or DNA modification activity, free of kinase activity, and/or free of phosphorylation and/or glycosylation activities, in each case, under desired test conditions (e.g., conditions of time, temperature, pH, salinity, model or intended substrate and/or others), for example, conditions intended to replicate conditions of a specific use of the H6 RNA polymerase composition or intended to represent conditions for a range of uses.

In some embodiments, H6 RNA polymerases (e.g., variant H6 RNA polymerases) and compositions comprising one or more H6 RNA polymerases (e.g., one or more variant H6 RNA polymerases) may have any desirable form including, for example, a liquid, a gel, a film, a powder, a cake, and/or any dried or lyophilized form. An H6 RNA polymerase composition may comprise an H6 RNA polymerase (e.g., a wt or variant H6 RNA polymerase) and a support or matrix, for example, a film, gel, fabric, column or bead comprising, for example, a magnetic material, agarose, polystyrene, polyacrylamide, and/or chitin. An H6 RNA polymerase (e.g., a wt or variant H6 RNA polymerase) and compositions comprising an H6 RNA polymerase (e.g., a wt or variant H6 RNA polymerase) may be active at higher temperatures. For example, an H6 RNA polymerase (e.g., a wt or variant H6 RNA polymerase) or an H6 RNA polymerase composition (e.g., comprising an H6 RNA polymerase or a variant H6 RNA polymerase) may display RNA synthesis activity at 30° C.-70° C., 37° C.-65° C., 40° C.-65° C., or 45° C.-65° C. that exceeds the activity of T7 RNA polymerase (wild type) with the same template under the same conditions. Aqueous compositions (e.g., comprising an H6 RNA polymerase or a variant H6 RNA polymerase) may include, for example, one or more elements that reduce the composition's melting temperature including, for example, DMSO, methanol, glycerol, ethylene glycol, propylene glycol, sugars, amino acids, and proteins among others.

In some embodiments, an H6 RNA polymerase (e.g., a wt or variant H6 RNA polymerase) may be encoded by a nucleic acid sequence that, when transcribed, translated, and/or processed, results in an amino acid sequence having at least 75%, at least 80%, at least 85%, at least 88%, at least 90%, at least 91%, at least 93%, at least 95%, at least 96%, at least 97%, at least 98% or at least 99% identity to one or more of SEQ ID NOS: 1-22. In some embodiments, a variant H6 RNA polymerase may be encoded by a nucleic acid sequence that, when transcribed, translated, and/or processed, results in an amino acid sequence according to Table 1, for example, an amino acid sequence having at least 75%, at least 80%, at least 85%, at least 88%, at least 90%, at least 91%, at least 93%, at least 95%, at least 96%, at least 97%, at least 98% or at least 99% identity to one or more of SEQ ID NOS: 1-20 and including one or more substitutions, insertions or deletions relative to SEQ ID NO: 21. A nucleic acid encoding a variant H6 RNA polymerase may be included in an expression cassette, expression vector, or other expressible form suitable for in vitro or in vivo expression (e.g., in E. coli or other bacteria or P. pastoris or other yeast). A nucleic acid encoding an H6 RNA polymerase (e.g., a wt or variant H6 RNA polymerase) may be modified or optimized (e.g., codon optimized) for expression in a desired organism or cell-free expression system.

### Methods and Workflows

The present disclosure further relates to methods and workflows that include an H6 RNA polymerase (e.g., a wt or variant H6 RNA polymerase). A method of producing an H6 RNA polymerase may comprise, for example, contacting (a) an H6 RNA polymerase transcript comprising an RNA encoding an amino acid sequence according to Table 1, for example, encoding an amino acid sequence having (i) at least 75%, at least 80%, at least 85%, at least 88%, at least 90%, at least 91%, at least 93%, at least 95%, at least 96%, at least 97%, at least 98% or at least 99% identity to one or more of SEQ ID NOS: 1-20, and (ii) at least one substitution at a position selected from positions corresponding to positions 106, 108, 134, 215, 232, 283, 327, 329, 378, 402, 419, 454, 459, 480, 481, 484, 490, 516, 519, 538, 715, 717, 756, 759, 798, 803, 848 of SEQ ID NO: 21 with (b) an expression system (e.g., a cell-based or cell-free expression system). An H6 RNA polymerase transcript may be capped or uncapped, according to some embodiments. Uncapped RNA may be synthesized using solid-phase oligonucleotide synthesis chemistry or by transcribing a DNA template using a polymerase (e.g., an H6 RNA polymerase) in an in vitro transcription reaction, for example. In some embodiments, a composition may comprise a capping enzyme, S-adenosyl methionine (SAM), and/or a cap 2′O methyltransferase enzyme (2′OMTase).

In some embodiments, an H6 RNA polymerase (e.g., a wt or variant H6 RNA polymerase) may be produced by contacting an H6 RNA polymerase protein expression DNA construct operably linked to an expression control sequence (e.g., an appropriate promoter) to an in vitro transcription/translation system such as PURExpress In vitro Protein Synthesis Kit (New England Biolabs, Inc.) or TnT Quick Coupled Transcription/Translation System (Promega). In addition, an H6 RNA polymerase (e.g., a wt or variant H6 RNA polymerase) can be produced by contacting an H6 RNA polymerase expression DNA construct under the control of an appropriate promoter to a cell-free protein synthesis system derived from organisms such as E. coli (e.g., NEBExpress Cell-free E. coli Protein Synthesis System (New England Biolabs, Inc.), rabbit, wheat germ, insect, or human. Reaction conditions (e.g., time, temperature, reaction composition) may be maintained or adjusted as needed to express the H6 protein. Expressed H6 protein may be purified by appropriate methods (e.g., chromatographic methods).

The present disclosure relates, in some embodiments, to methods for making an RNA of interest. An RNA of interest may be any RNA molecule including, for example, non-naturally occurring RNA, viral RNA, prokaryotic RNA, eukaryotic RNA, and/or archaeal RNA. An RNA of interest may be a messenger RNA (mRNA), ribosomal RNA (rRNA), transfer RNA (tRNA), small RNA (sRNA), microRNA (miRNA), long noncoding RNA (lncRNA), circular RNA (circRNA), aptamer RNA, antisense RNA, silencing RNA (siRNA), guide RNA (gRNA), or any combination thereof. An RNA of interest may be itself a therapeutic RNA or may be included in a therapeutic RNA composition. A method may comprise, for example, contacting a template DNA (or RNA) encoding the RNA of interest with an H6 RNA polymerase (e.g., a wild type or a variant according to Table 1) to produce the RNA of interest. A template may comprise an H6 promoter (e.g., a sequence having ≥70%, ≥75%, ≥80%, ≥85%, or ≥90% identity to SEQ ID NO: 23) operably linked to the coding sequence for the RNA of interest. Contacting may include contacting at temperatures in ranges X to Y, where X is any of 30° C., 35° C., 37° C., 40° C., 42° C., 45° C., 50° C. and Y is any of 45° C., 50° C., 55° C., 60° C., 65° C., 66° C., 68° C., 70° C. and X<Y. For example, contacting the polymerase and template may comprise contacting the two at a temperature in a range of 30° C.-70° C., 37° C.-65° C., 40° C.-65° C., or 45° C.-65° C. Contacting may further comprise suitable conditions for RNA synthesis including, for example, contacting the temple, polymerase, NTPs and optionally one or more modified NTPs. Contacting may further comprise contacting one or more of the foregoing in a composition comprising a buffer and/or having a pH in a range from X^{†} to Y^{†}, where X^{†} is any of pH 4, 4.5, 5, 5.5, 6, 6.5, 7, and Y^{†} is any of pH 6.5, 7, 7.5, 8, 8.5, 9, 9.5, 10, 10.5, 11 and X^{†}<Y^{†}. For example, a composition may have a pH from 6-9, 6.5-8.5 or 7-8. In some embodiments, a method may comprise contacting an H6 RNA polymerase (e.g., a wt or variant H6 RNA polymerase), a polynucleotide template (e.g., DNA or RNA) comprising a sequence encoding an RNA of interest (e.g., a therapeutic RNA), one or more NTPs, one or more modified NTPs, a buffer, and a salt (e.g., MgCl₂) at a temperature in a range of 30° C.-70° C., 37° C.-65° C., 40° C.-65° C., or 45° C.-65° C. and a pH in a range of 6-9, 6.5-8.5 or 7-8.

According to some embodiments, a method may further include capping the transcript, for example, by contacting the transcript and a capping enzyme (e.g., a Vaccinia capping enzyme, a Faustovirus capping enzyme). RNA transcript produced by an H6 RNA polymerase (e.g., a wt or variant H6 RNA polymerase) may comprise a smaller fraction of double-stranded RNA than similar RNA transcript produced by, for example, T7 RNA polymerase. For example, an H6 RNA polymerase may produce RNA transcript having less than ½, less than ⅓, less than ¼, or less than 1/10 the double stranded RNA found in the same total quantity of RNA transcript produced by T7 RNA polymerase as measured, for example, by the CE and LC-MS methods of Examples 4 and 5 or by antibodies specific for dsRNA. Example methods may be found in U.S. Pat. No. 10,034,951. In some embodiments, RNA transcript produced by an H6 RNA polymerase (e.g., a wt or variant H6 RNA polymerase) may be less immunostimulatory than a like amount of a similar RNA transcript produced by, for example, T7 RNA polymerase. For example, an H6 RNA polymerase may produce RNA transcript having less than ½, less than ⅓, less than ¼, or less than 1/10 the immunostimulatory activity of a like amount of a the same RNA transcript produced by T7 RNA polymerase as measured by, for example, interferon and/or cytokine expression by mammalian cells following exposure to such transcripts. RNA transcript produced by an H6 RNA polymerase (e.g., a wt or variant H6 RNA polymerase) may have increased homogeneity at the 3′ end of the transcript. For example, an H6 RNA polymerase may produce an RNA transcript for which the majority of the RNA species have the same nucleotides at the 3′ end, and this proportion of RNA species with the same 3′ end may be ≥2×, ≥5×, ≥8×, ≥10×, ≥12×, ≥15×, ≥18×, ≥20× higher than a similar RNA transcript produced by, for example, T7 RNA polymerase. Example methods may be found in U.S. Pat. No. 10,034,951.

The present disclosure provides, in some embodiments, nucleic acid sequence based amplification (“NASBA”) methods using an H6 RNA polymerase described herein. NASBA methods may be used as a rapid diagnostic test for pathogenic (among other) RNA viruses (e.g., influenza A, foot-and-mouth disease virus, severe acute respiratory syndrome (SARS)-associated coronavirus, HIV-1, human bocavirus (HBOV)) and also parasites like Trypanosoma brucei. NASBA methods are isothermal, often run at a constant temperature of at least 41° C. Use of an H6 RNA polymerase (e.g., wildtype or a variant thereof) permits increasing the reaction temperature (e.g., to 45° C. or higher, 50° C. or higher, 55° C. or higher, or 60° C. or higher). In some embodiments, an NASBA method comprises contacting an H6 RNA polymerase, an RNA template, and a primer containing a promoter sequence wherein the primer hybridizes to a complementary site at the 3′ end of the template, and reverse transcriptase synthesizes the opposite, complementary DNA strand. RNAse H destroys the RNA template rom the DNA-RNA hybrid, and a second primer hybridizes to the 5′ end of the cDNA strand. The second primer is extended using the cDNA as a template, resulting in double stranded DNA. An H6 RNA polymerase may continuously produce complementary RNA strands of this template, which results in amplification. The amplicons are antisense to the original RNA template. A higher incubation temperature results in less non-specific binding of DNA primers to the RNA. In some embodiments, the reaction may include a temperature-sensitive inhibitor of the polymerase, thereby allowing the polymerase to remain inactive until the temperature rises.

The present disclosure provides, in some embodiments, transcription-mediated amplification (“TMA”) methods using an H6 RNA polymerase described herein. TMA methods may be performed as isothermal, single-tube nucleic acid amplifications using two enzymes, an H6 RNA polymerase and reverse transcriptase, to rapidly amplify a target RNA/DNA. TMA may be configured to provide simultaneous detection of multiple pathogenic organisms in a single tube, allowing, for example, clinical laboratories to perform nucleic acid test (NAT) assays for blood screening with fewer steps, less processing time, and faster results. It may be used in molecular biology, forensics, and medicine for the rapid identification and diagnosis of pathogenic organisms. In contrast to similar techniques such as polymerase chain reaction and ligase chain reaction, this method involves RNA transcription (via an RNA polymerase) and DNA synthesis (via reverse transcriptase) to produce an RNA amplicon (the source or product of amplification) from a target nucleic acid. This technique can be used for both target RNA and DNA.

NASBA and TMA reactions performed at a higher temperature (e.g., in the range of 45° C. to 60° C.) produce less non-specific amplification products relative to the same reactions that are performed at a lower temperatures (e.g., 41° C.). DMSO or other compounds (e.g, betaine, ethylene glycol and 1,2-5 propanediol, etc.) that lower the melting temperature of the primers and substrate DNA or RNA may be unnecessary in the amplification reaction at a higher temperature using an H6 RNA polymerase (e.g., wildtype or a variant thereof).

A method of making an RNA of interest may further comprise contacting the produced RNA with a one or more pharmaceutically acceptable additives (e.g., excipients, diluents, and/or carriers), including, for example, fluids, solvents, dispersion media, wetting agents, crowding agents, micelles, lipidoids, liposomes, polymers, lipoplexes, peptides, proteins, salts, surface active agents, isotonic agents, thickeners, emulsifiers, preservatives, stabilizers, solubilizers, buffers, sugars, starches, cellulose, waxes, glycols, polyols, polyesters, polycarbonates, polyanhydrides, hyaluronidase, nanoparticles (e.g., lipid nanoparticles, core-shell nanoparticles, and/or nanoparticle mimics), and combinations thereof. In some embodiments, pharmaceutically acceptable additives protect, preserve, and/or stabilize an RNA of interest during manufacture, storage, use, and/or administration to a subject. Examples of pharmaceutical acceptable additives include those described in U.S. Patent Publication No. 2017/0119740. A method of making an RNA of interest may further comprise contacting the RNA with one or more additives selected from lipidoids, liposomes, polymers, lipoplexes, peptides, proteins, cells transfected with HCMV RNA vaccines (e.g., for transplantation into a subject), hyaluronidase, nanoparticles (e.g., lipid nanoparticles, core-shell nanoparticles, and/or nanoparticle mimics).

Manufactured RNAs may be formulated for delivery and/or delivered to a eukaryotic organism. Examples of subjects that may receive a manufactured RNA include humans and non-human animals (e.g., mouse, rat, rabbit, dog, cat, cattle, swine, sheep, horse or primate). Manufactured RNAs may be delivered to plants or plant cells, according to some embodiments, to confer or augment resistance to or tolerance of an environmental condition (e.g., drought, salt) and/or to prevent, mitigate or treat herbivory, pathogen infection, or the effects thereof. Manufactured RNA also may be delivered to one or more yeast cells.

In some embodiments, the present disclosure provides methods for preparing an RNA dosage form comprising, contacting an H6 RNA polymerase and a transcript encoding the RNA of interest to produce a transcribed RNA, optionally capping the transcribed RNA with a capping enzyme to form a capped RNA, and contacting produced RNA or the capped RNA with one or more pharmaceutically acceptable additives, binders, buffers, coatings, colors, controlled release agents, delivery agents (e.g., liposomes, propellants), diluents, disintegrants, dyes, excipients, fillers, lipids, lubricants, salts, sorbants, stabilizers, and/or other agents to produce an RNA dosage form. An RNA of interest may be combined with (e.g., in a single dosage form) or delivered concurrently or in sequence with one or more other active pharmaceutical agents. An RNA and/or its encoded translation product(s) may function in a subject as an active pharmaceutical agent, according to some embodiments. An RNA (e.g., a capped RNA dosage form) may be administered by any suitable route of administration, including transdermal, oral, enteral, parenteral, ocular, ottic, transmucosal, sublingual, and pulmonary (e.g., by nebulization and/or inhalation) routes, and combinations thereof.

An RNA of interest can either be naked or formulated in a suitable form for delivery to a subject, e.g., a human. Formulations can include liquid formulations (solutions, suspensions, dispersions), topical formulations (gels, ointments, drops, creams), liposomal formulations (such as those described in: U.S. Pat. No. 9,629,804 B2; US 2012/0251618 A1; WO 2014/152211; US 2016/0038432 A1). The cells into which the RNA product is introduced may be in vitro (i.e., cells that have been cultured in vitro on a synthetic medium). Accordingly, the RNA product may be transfected into the cells. The cells into which the RNA product is introduced may be in vivo (cells that are part of a mammal). The cells into which the RNA product is introduced may be present ex vivo (cells that are part of a tissue, e.g., a soft tissue that has been removed from a mammal or isolated from the blood of a mammal).

### Kits

The present disclosure further relates to kits including an H6 RNA polymerase (e.g., a wt or variant H6 RNA polymerase). For example, a kit may include an H6 RNA polymerase (e.g., a wt or variant H6 RNA polymerase) and NTPs, other enzymes (e.g., other polymerases, capping enzymes, others), buffering agents, or combinations thereof. Enzymes may be included in a storage buffer. Any suitable storage buffer may be used, for example, buffers comprising one or more of a cryoprotectant (e.g., a polyol such as glycerol, an antifreeze protein), a salt, a detergent, a reducing agent, a sugar, a chelator, and an antimicrobial agent and having a pH tolerated by the enzyme to be stored, for example, between pH 6 and 9. A composition or kit may include a reaction buffer which may be in concentrated form, and the buffer may contain additives (e.g. glycerol), salt (e.g. NaCl, KCl), reducing agent, EDTA or detergents, among others. Detergents include nonionic detergents (e.g., t-octylphenoxypolyethoxyethanol), anionic detergents (e.g., alkylbenzene sulfonates), cationic detergents (e.g., alkylbenzene quaternary ammonium), and zwitterionic detergents. A composition or kit comprising rNTPs may include one, two, three of all four of rATP, rUTP, rGTP and rCTP. A kit may further comprise one or more modified nucleotides. A kit may optionally comprise one or more primers (random primers, bump primers, exonuclease-resistant primers, chemically-modified primers, custom sequence primers, or combinations thereof).

A kit may be a non-natural collection of components configured, for example, for convenient storage, shipping, delivery, and/or use. One or more components of a kit may be included in one container for a single step reaction, or one or more components may be contained in one container, but separated from other components for sequential use or parallel use. The contents of a kit may be formulated for use in a desired method or process.

A kit is provided that contains: (i) an H6 RNA polymerase (e.g., a wt or variant H6 RNA polymerase); and (ii) a buffer. An H6 RNA polymerase (e.g., a wt or variant H6 RNA polymerase) may have a lyophilized form or may be included in a buffer (e.g., a storage buffer or a reaction buffer in concentrated form). A kit may contain an H6 RNA polymerase (e.g., a wt or variant H6 RNA polymerase) in a mastermix suitable for receiving and amplifying a template nucleic acid. An H6 RNA polymerase (e.g., a wt or variant H6 RNA polymerase) may be a purified enzyme so as to contain substantially no DNA or RNA and no nucleases. The reaction buffer in (ii) and/or storage buffers containing the RNA polymerase in (i) may include a non-ionic surfactant, an ionic surfactant (e.g. an anionic or zwitterionic surfactant) and/or a crowding agent. A kit may include an H6 RNA polymerase (e.g., a wt or variant H6 RNA polymerase) and the reaction buffer in a single tube or in different tubes.

A subject kit may further include instructions for using the components of the kit to practice a desired method. The instructions may be recorded on a suitable recording medium. For example, instructions may be printed on a substrate, such as paper or plastic, etc. As such, the instructions may be present in the kits as a package insert, in the labeling of the container of the kit or components thereof (i.e., associated with the packaging or subpackaging) etc. Instructions may be present as an electronic storage data file residing on a suitable computer readable storage medium (e.g. a CD-ROM, a flash drive). Instructions may be provided remotely using, for example, cloud or internet resources with a link or other access instructions provided in or with a kit.

### Embodiments

Embodiments disclosed herein include:

1. An H6 RNA polymerase having an amino acid sequence at least 95% identical to SEQ ID NO: 21.
2. A variant H6 RNA polymerase having (a) an amino acid sequence at least 98% identical to any of SEQ ID NOS: 1-20, and (b) at least one substitution relative to SEQ ID NO: 21.
3. A variant H6 RNA polymerase having (a) an amino acid sequence at least 95% identical to any of SEQ ID NOS: 1-20, and (b) at least one substitution relative to SEQ ID NO: 21 at a position selected from positions corresponding to positions 106, 108, 134, 215, 232, 283, 327, 329, 378, 402, 419, 454, 459, 480, 481, 484, 490, 516, 519, 538, 715, 717, 756, 759, 798, 803, and 848 of SEQ ID NO: 21.
4. A variant H6 RNA polymerase having (a) an amino acid sequence at least 95% identical to any of SEQ ID NOS: 1-20, and (b) at least one substitution relative to SEQ ID NO: 21 at a position selected from positions corresponding to positions 215, 459, 538, 759, 798, 803, and 848 of SEQ ID NO: 21.
5. A variant H6 RNA polymerase according to Embodiment 2 or Embodiment 3 or Embodiment 4 further comprising a second substitution at a position (i) other than the position of the first substitution and (ii) corresponding to positions 215, 459, 538, 759, 798, 803, and 848 of SEQ ID NO: 21.
6. A variant H6 RNA polymerase according to Embodiment 5 further comprising a third substitution at a position (i) other than the position of the first and second substitutions and (ii) corresponding to positions 215, 459, 538, 759, 798, 803, and 848 of SEQ ID NO: 21.
7. A variant H6 RNA polymerase having (a) an amino acid sequence at least 95% identical to any of SEQ ID NOS: 1-20, and (b) substitutions relative to SEQ ID NO: 21 at positions corresponding to positions 215, 459, 538, 759, 798, 803, and 848 of SEQ ID NO: 21.
8. A fusion comprising, in an N-terminal to C-terminal direction, (I) a purification tag or a sorting signal peptide, and (II) an H6 RNA polymerase according to any preceding Embodiment operably linked to (I).
9. A fusion comprising, in an N-terminal to C-terminal direction, (III) an H6 RNA polymerase according to Embodiment 1, 2, 3, 4, 5, 6, or 7, and (IV) a purification tag or a sorting signal peptide operably linked to (III).
10. A composition comprising an H6 RNA polymerase according to Embodiment 1, 2, 3, 4, 5, 6, or 7 or a fusion according to Embodiment 8 or 9.
11. A composition according to Embodiment 10 further comprising a template encoding an RNA of interest.
12. A composition according to Embodiment 11, wherein the template further comprises an H6 RNA polymerase promoter having the sequence of SEQ ID NO: 23.
13. A composition according to Embodiment 10, 11, or 12 further comprising at least one of a buffering agent and a polyamine.
14. A composition according to Embodiment 13, wherein the composition comprises both the buffering agent and the polyamine.
15. A composition according to Embodiment 13 or 14, wherein the buffering agent comprises HEPES, MES, MOPS, TAPS, tricine, Tris, ACES, ADA, BES, Bicine, CAPS, carbonic acid/bicarbonic acid, CHES, citric acid, DIPSO, EPPS, histidine, MOPSO, phosphoric acid, PIPES, POPSO, TAPS, TAPSO, or triethanolamine.
16. A composition according to Embodiment 13 or 14, wherein the polyamine comprises spermidine, spermine, putrescine, polyethylenimine, 1,4,7-triazacyclononane, cyclen, ethylenediamine, or 1,3,5,-triazinane.
17. A method of making an RNA of interest, the method comprising:
contacting:
   an H6 RNA polymerase according to Embodiment 1 or a variant H6 RNA polymerase according to any of Embodiments 2-7;
   a template encoding the RNA of interest;
   optionally, one or more NTPs;
   optionally, one or more modified NTPs; and
   optionally, a buffer,
to produce the RNA of interest, wherein the contacting is at a temperature in a range of 45° C.-65° C. and/or for a time in the range of seconds to hours and/or the RNA of interest is a therapeutic RNA.
   18. A method according to Embodiment 17, wherein the template further comprises an H6 RNA polymerase promoter having the sequence of SEQ ID NO: 23.
   19. A method according to Embodiment 17, wherein the RNA of interest comprises a capped RNA and contacting further comprising contacting the polymerase, the template, the optional components, if present, and a capping enzyme to produce the capped RNA.
   20. A method according to Embodiment 17, 18 or 19 further comprising contacting the RNA of interest with one or more pharmaceutically acceptable additives.
   21. A kit comprising:
an H6 RNA polymerase according to any of Embodiments 1-8 in a storage buffer; and
one or more NTPs.
   22. A kit according to Embodiment 21 further comprising one or more modified NTPs.
   23. A kit according to Embodiment 21 or 22 further comprising a capping enzyme.
   24. A kit according to Embodiment 21, 22 or 23 further comprising a reaction buffer, the reaction buffer comprising a buffering agent and a polyamine.
   25. A kit according to Embodiment 24, wherein the buffering agent comprises HEPES, MES, MOPS, TAPS, tricine, Tris, ACES, ADA, BES, Bicine, CAPS, carbonic acid/bicarbonic acid, CHES, citric acid, DIPSO, EPPS, histidine, MOPSO, phosphoric acid, PIPES, POPSO, TAPS, TAPSO, or triethanolamine.
   26. A kit according to Embodiment 24, wherein the polyamine comprises spermidine, spermine, putrescine, polyethylenimine, 1,4,7-triazacyclononane, cyclen, ethylenediamine, or 1,3,5,-triazinane.

### Examples

Some specific example embodiments may be illustrated by one or more of the examples provided herein.

### Example 1: Preparation of H6 and Hi-H6 RNA Polymerases

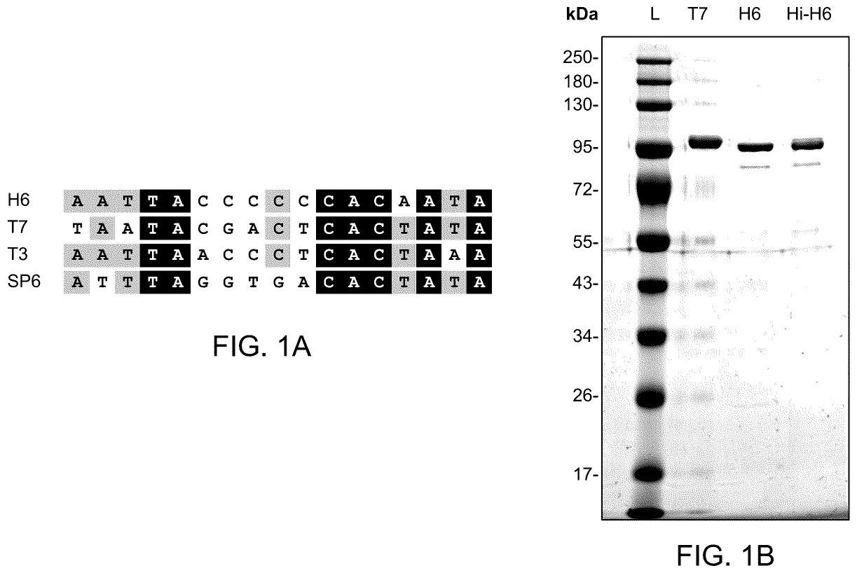

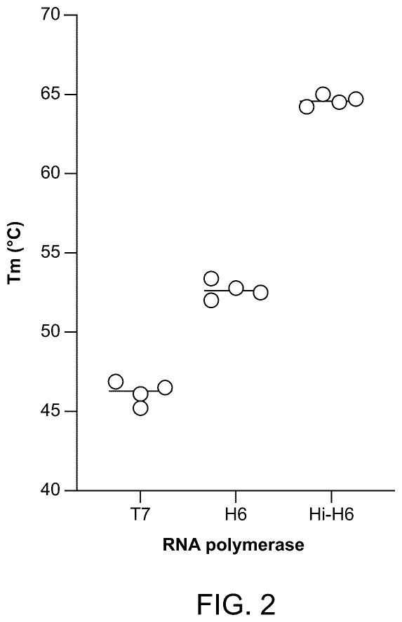

Sequences encoding proteins were cloned into a pET28 vector (Novagen. Inc) and expressed in T7-express E. coli cells (NEB, C2566). Overexpressed proteins were purified following standard protocol using a HisTrap column (Cytiva, 17524701). Fractions containing proteins were pooled and further purified using a HiTrap Heparin column (Cytiva, 1704601). Fractions containing purified proteins were pooled and dialyzed against a glycerol-free storage buffer or a glycerol storage buffer. Any suitable storage buffer may be used, for example, buffers comprising one or more of a cryoprotectant (e.g., glycerol or other polyols, antifreeze proteins), a salt, a detergent, a reducing agent, a sugar, a chelator, and an antimicrobial agent and having a pH tolerated by the enzyme to be stored, for example, between pH 6 and 9. FIG. 1A show promoter sequences evaluated. FIG. 1B shows example results of expression and purification, wherein the respective polymerase preparations appear to be nearly homogeneous.

### Example 2: Thermostability of T7, H6 and Hi-H6 RNA Polymerases

RNA polymerases were diluted with storage buffer to final concentration 20 ng/μL, 30 ng/μL, 40 ng/μL and 50 ng/μL. DSF experiments were carried out using Prometheus NT48 (NanoTemper). Fluorescence at 330 nm and 350 nm were measured at a heating ramp of 1.5° C. per minute from 25° C. to 90° C. The first inflection points of ratio of 350 nm/330 nm are reported as melting temperatures.

### Example 3: In Vitro Transcription

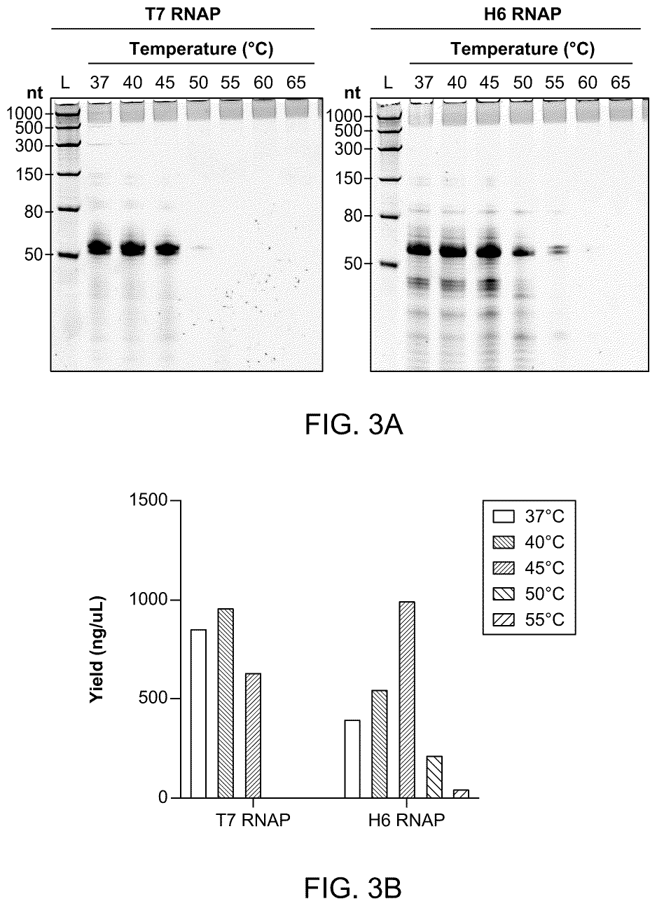

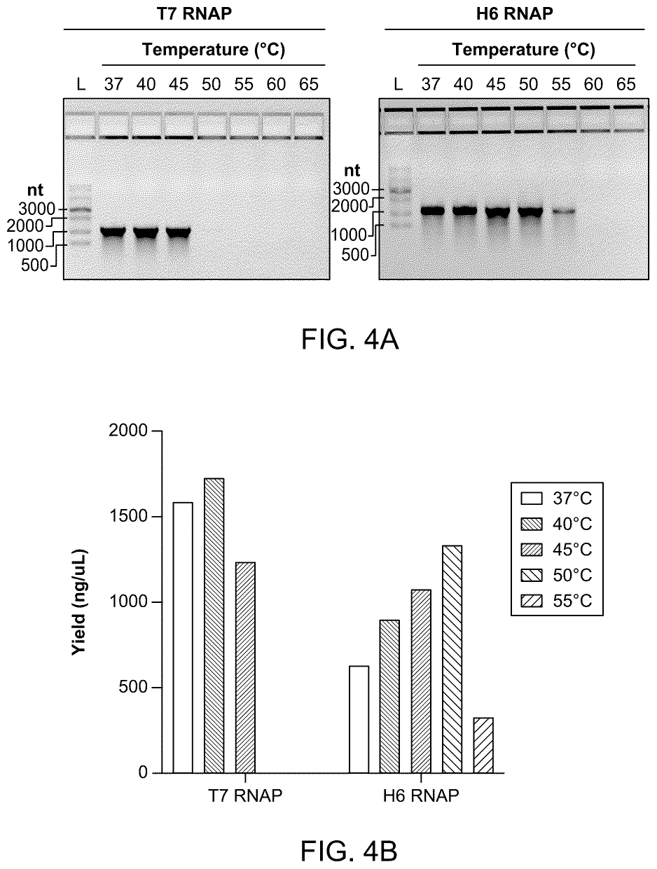

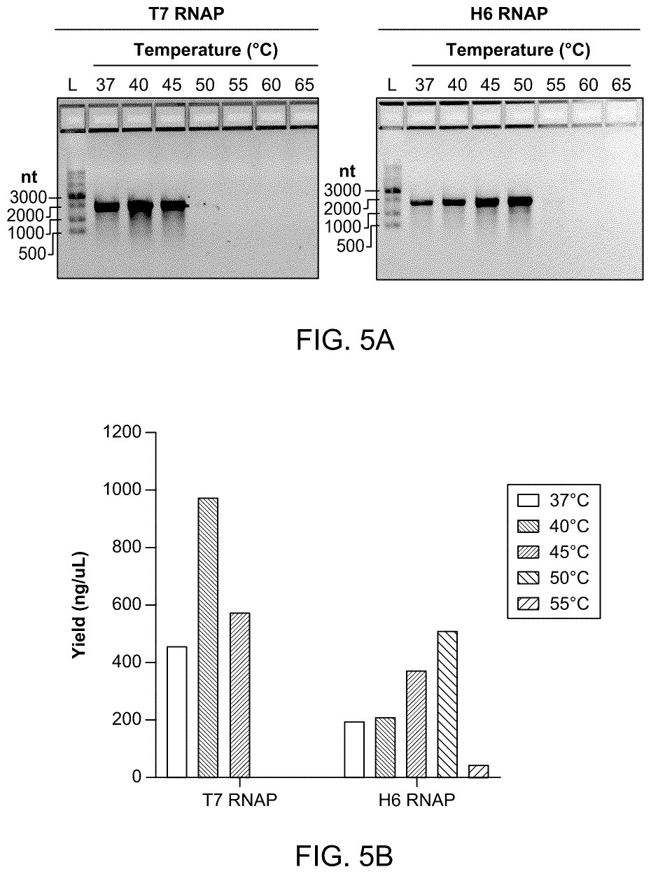

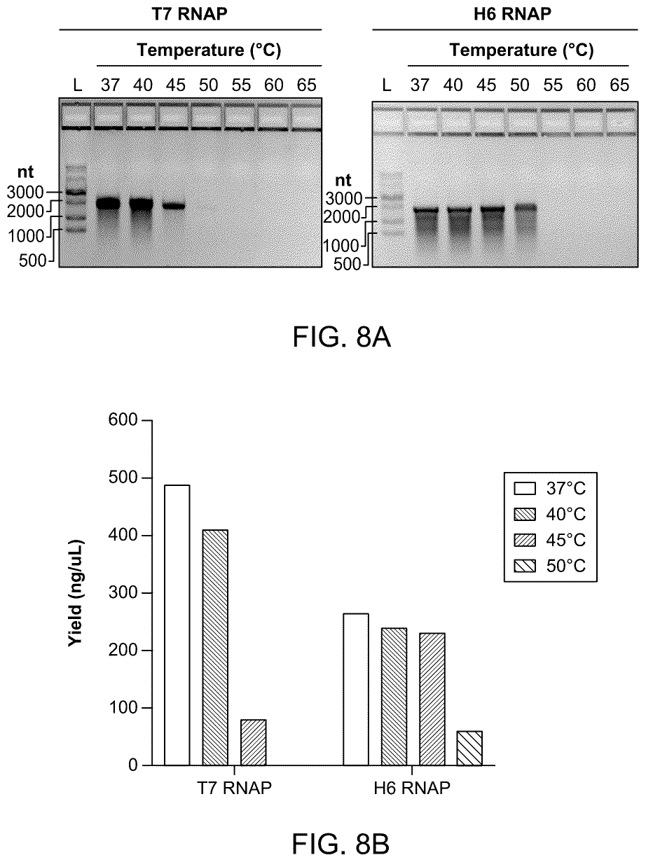

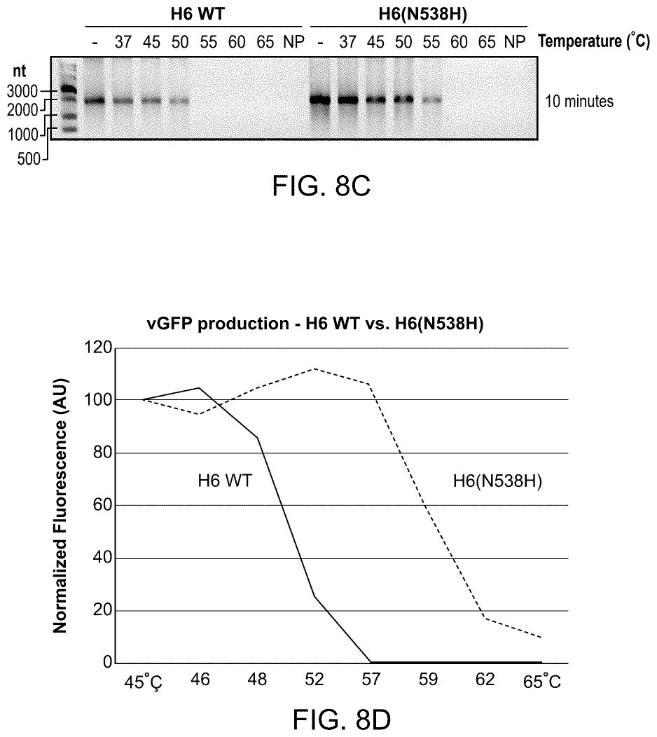

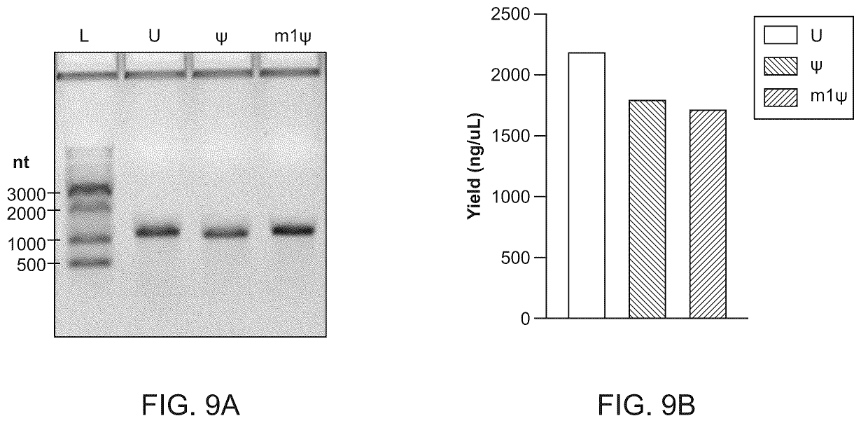

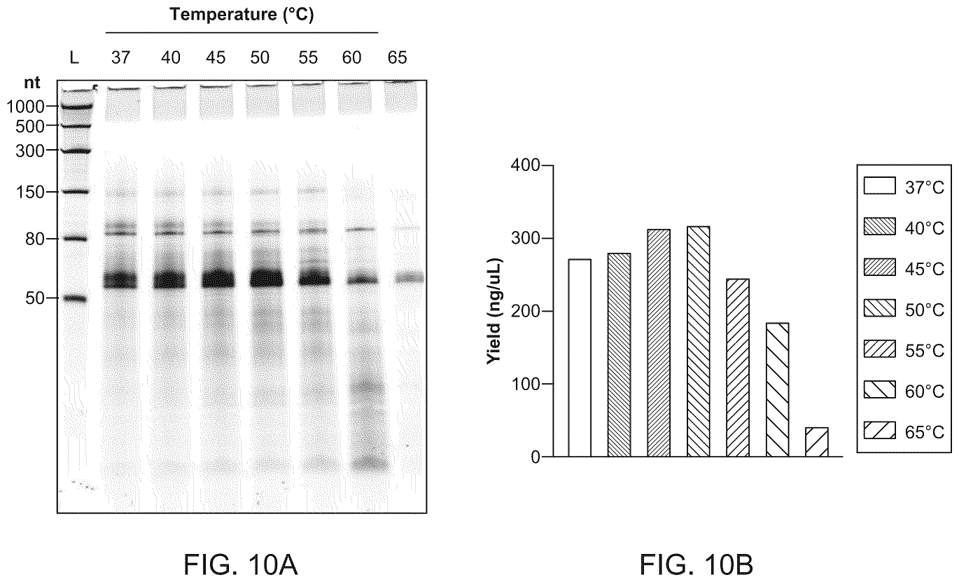

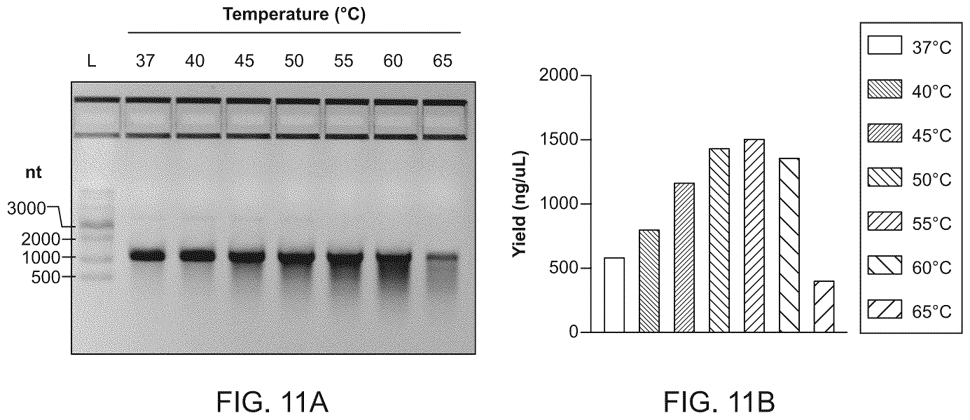

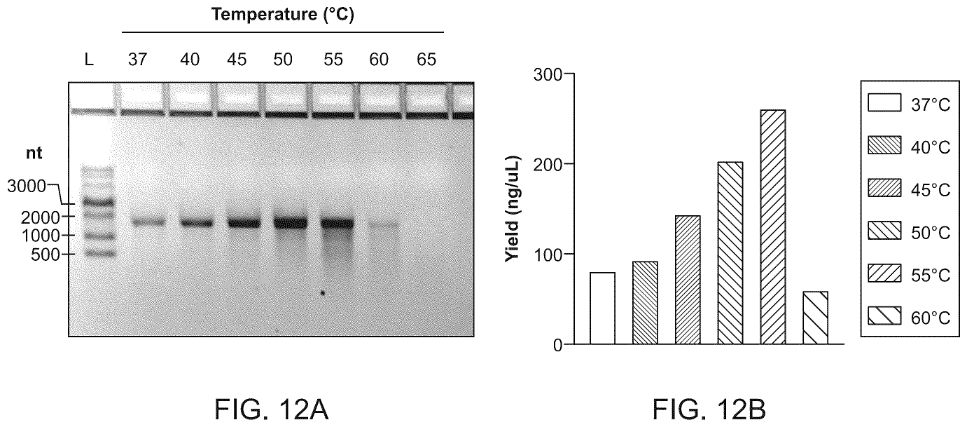

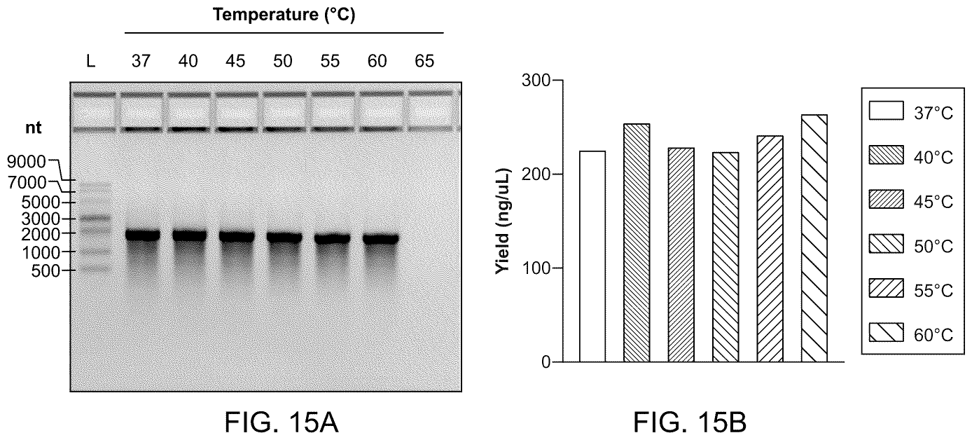

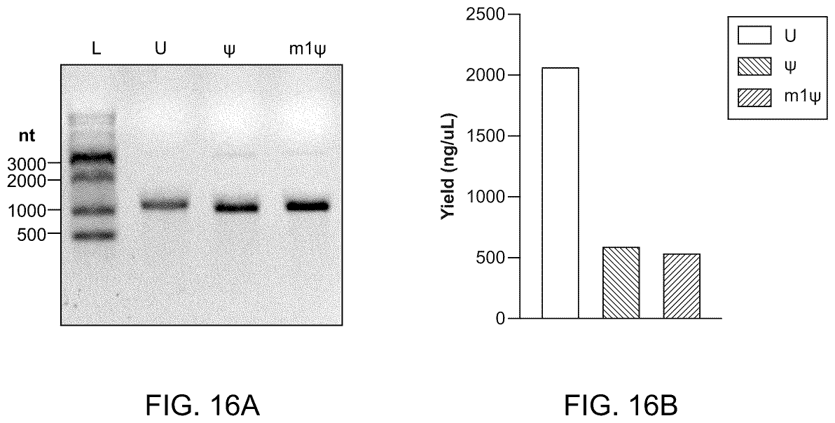

A typical 20-μL in vitro transcription was carried out with 20 ng/μL RNA polymerase, 10 ng/μL linearized DNA template or 1 μM short annealed oligonucleotides for the 50-mer or 10 ng/μL linearized DNA template for RNA1 or CLuc, 0.05 unit inorganic pyrophosphatase (NEB, M2403), 20 unit RNase Inhibitor (NEB, M0314) in reaction buffer containing 5 mM each NTP, 40 mM Tris-HCl pH 7.9, 20 mM MgCl₂, 1 mM DTT, and 2 mM spermidine. After an hour incubation at indicated temperatures, 2 units of DNase Turbo (Themo Fisher Scientific, AM 2238) were added to digest the DNA template at 37° C. for 30 min. RNA1, CLuc, and the 50 mer RNA were purified using Monarch RNA clean-up kit (NEB, T2040). For the modified nucleotide incorporation, the same procedures were performed except that UTP was completely replaced with pseudouridine-5′-Triphosphate (ψTP), TriLink Technologies, N1019) or N1-methylpseudouridine-5′-Triphosphate (m1ψTP), TriLink Technologies, N1081). RNA concentration was measured using Qubit RNA BR assay (Thermo Fisher Scientific, Q10210) or NanoDrop spectrophotometer (Thermo Fisher Scientific). The integrity of RNA was assessed using either 7 M urea 15% polyacrylamide gels, or 1.2% agarose gels. Example results are shown in FIGS. 3A, 3B, 4A, 4B, 5A, 5B, 8A, 8B, 8C, 8D, 9A, 9B, 10A, 10B, 11A, 11B, 12A, 12B, 15A, 15B, 16A, and 16B.

### Example 4: Mass Spectrometry Analysis

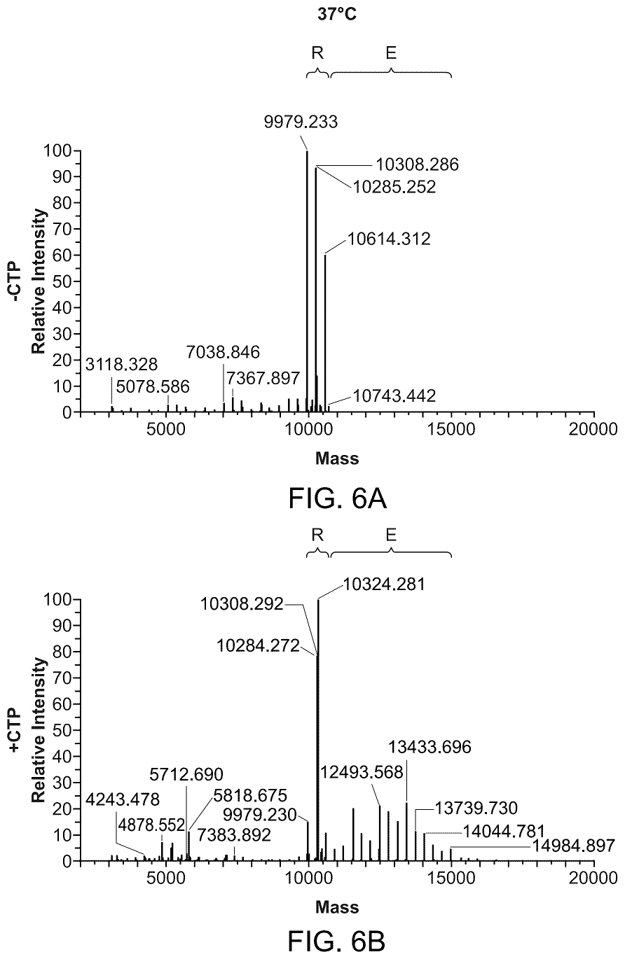

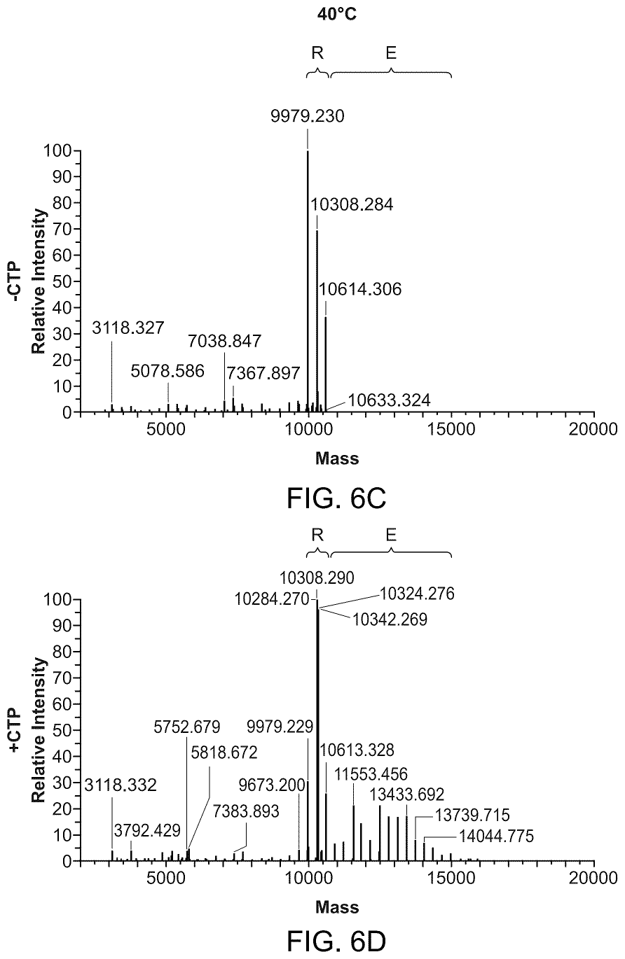

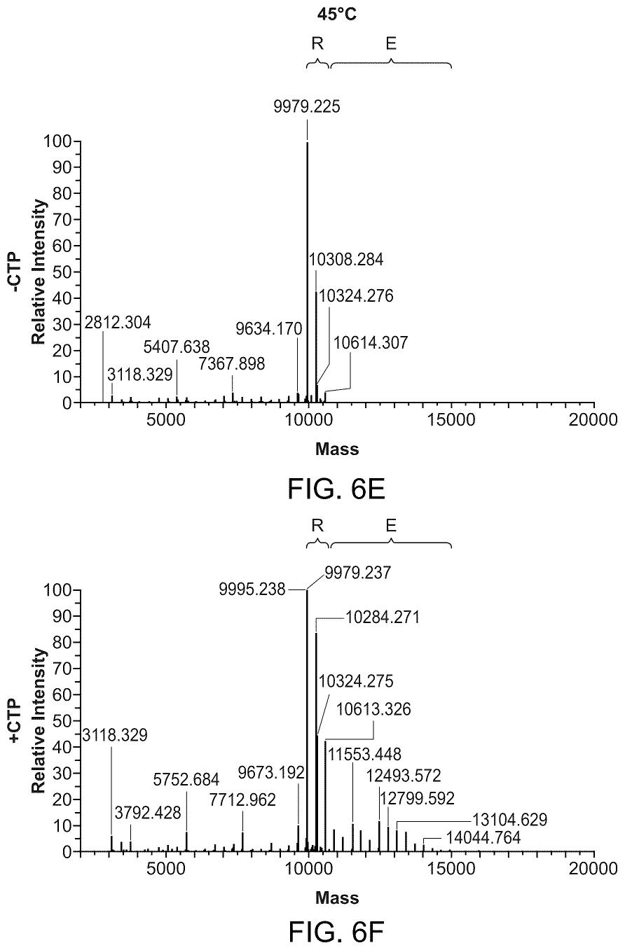

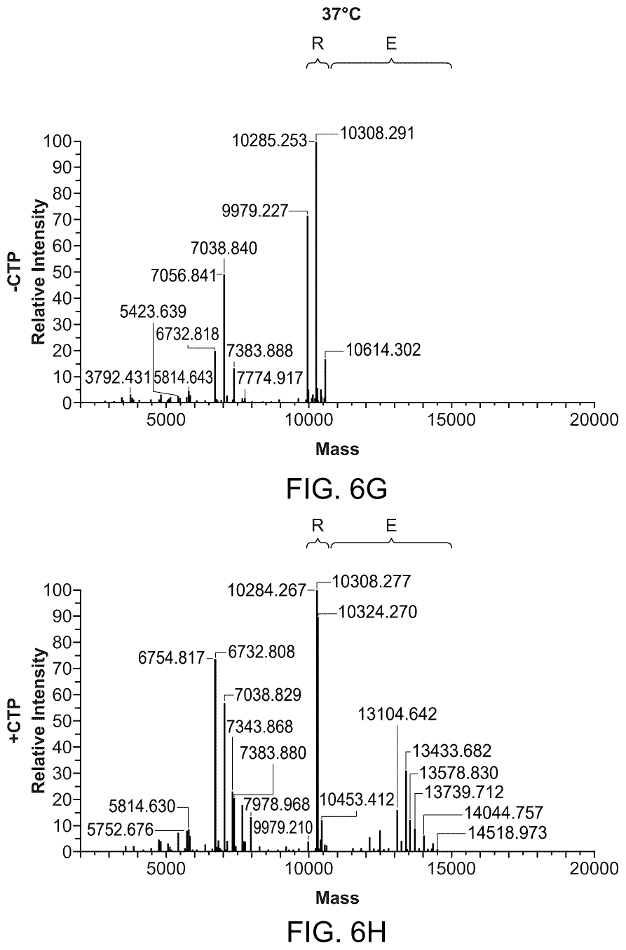

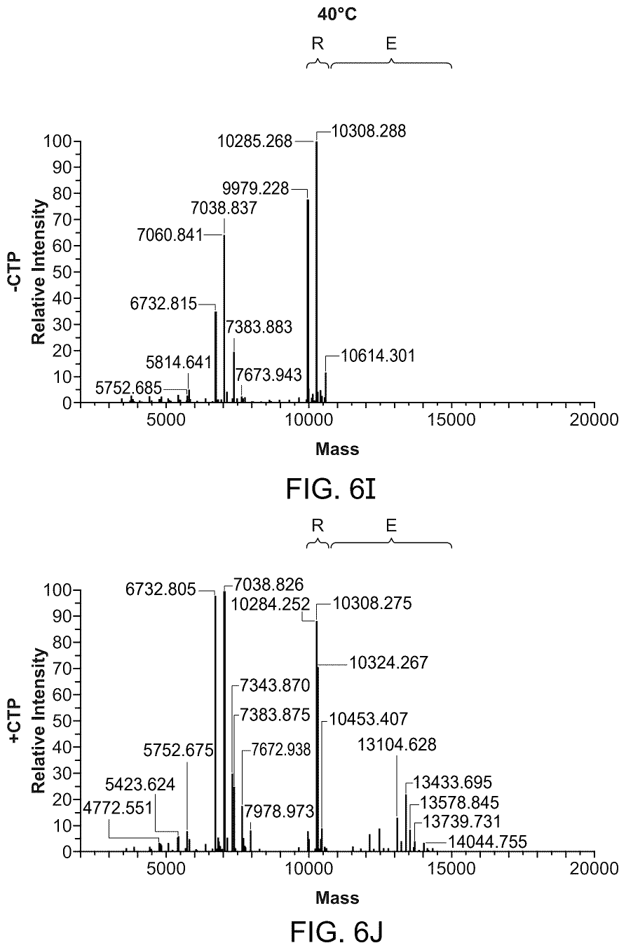

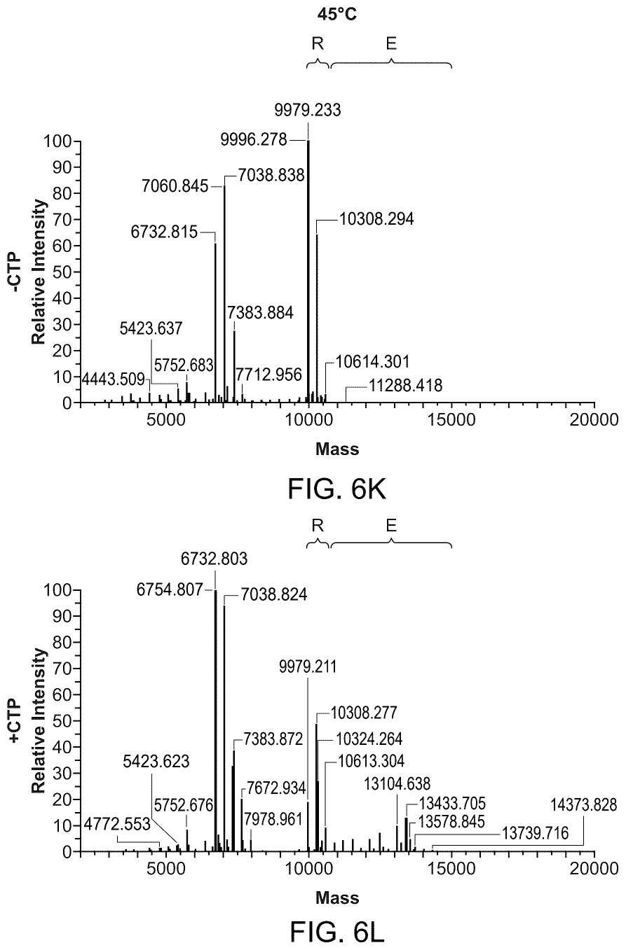

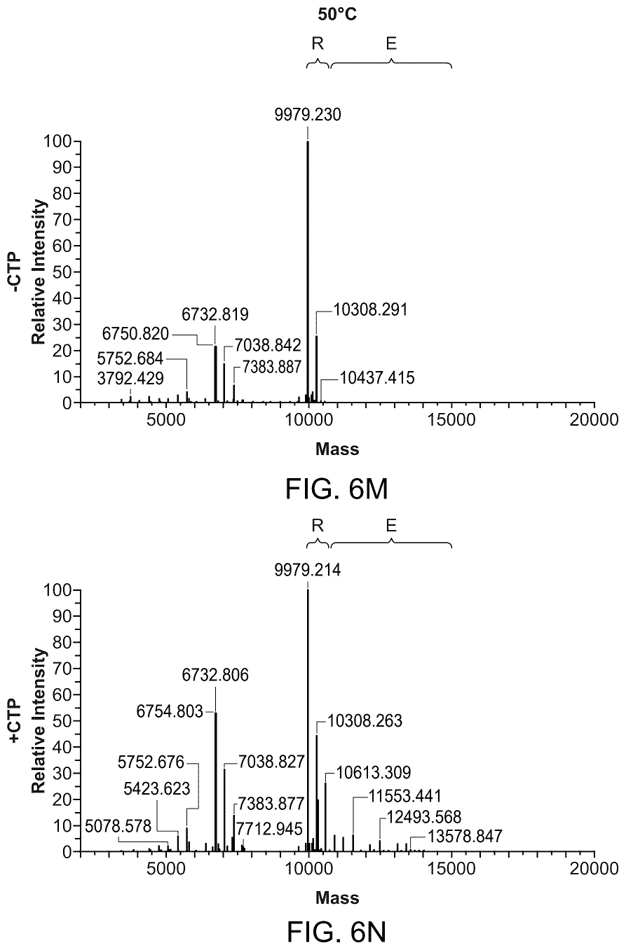

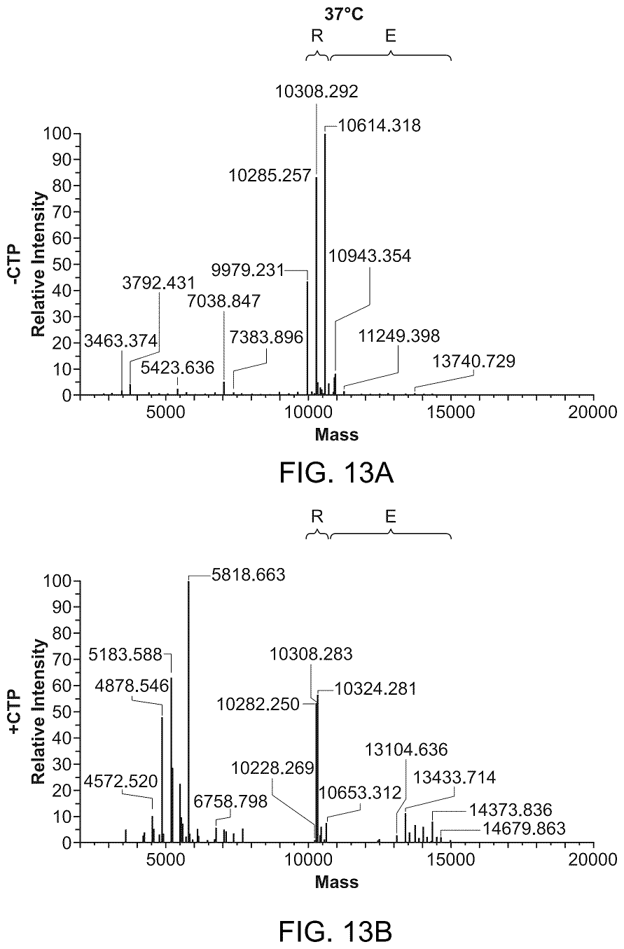

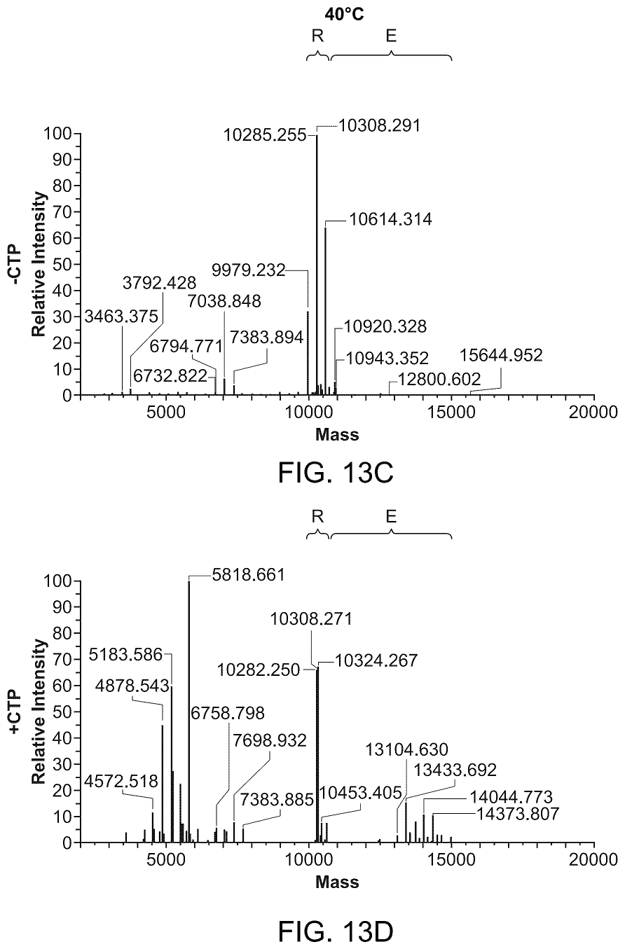

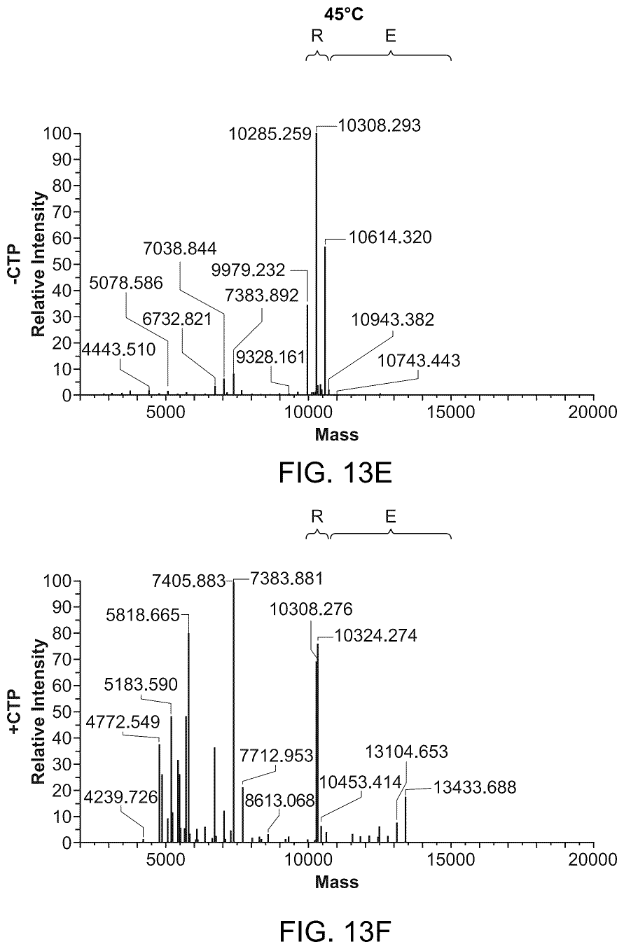

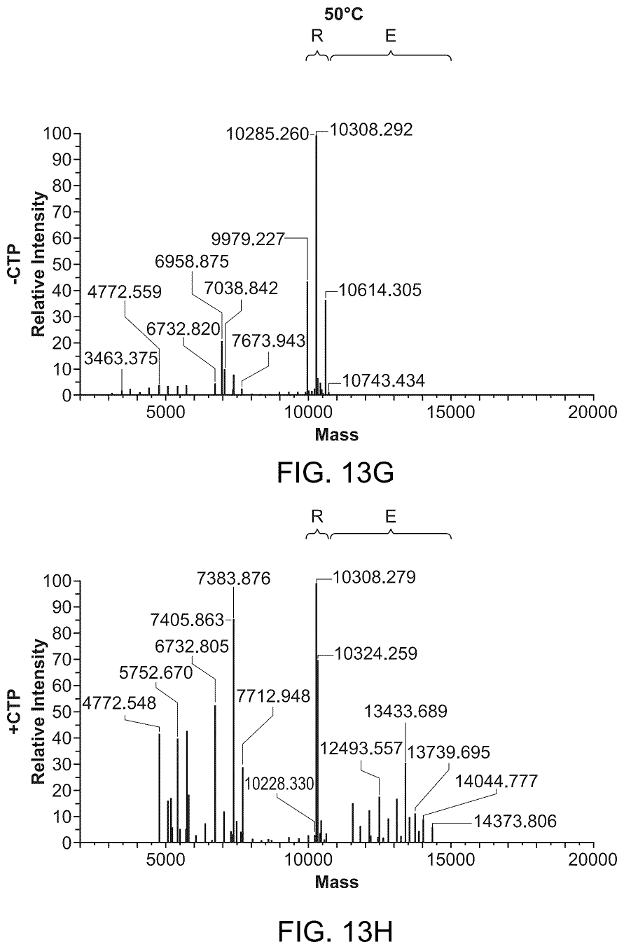

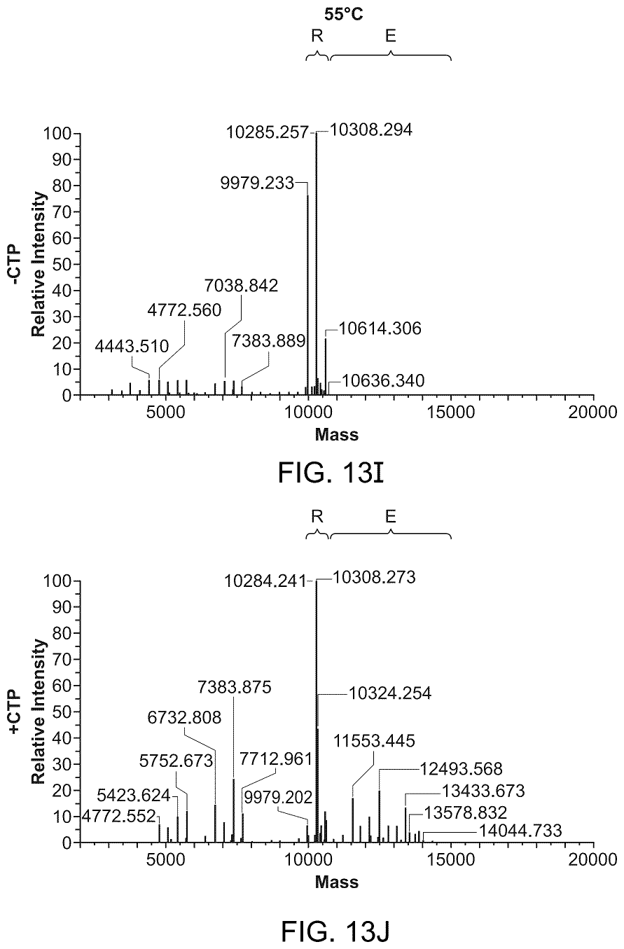

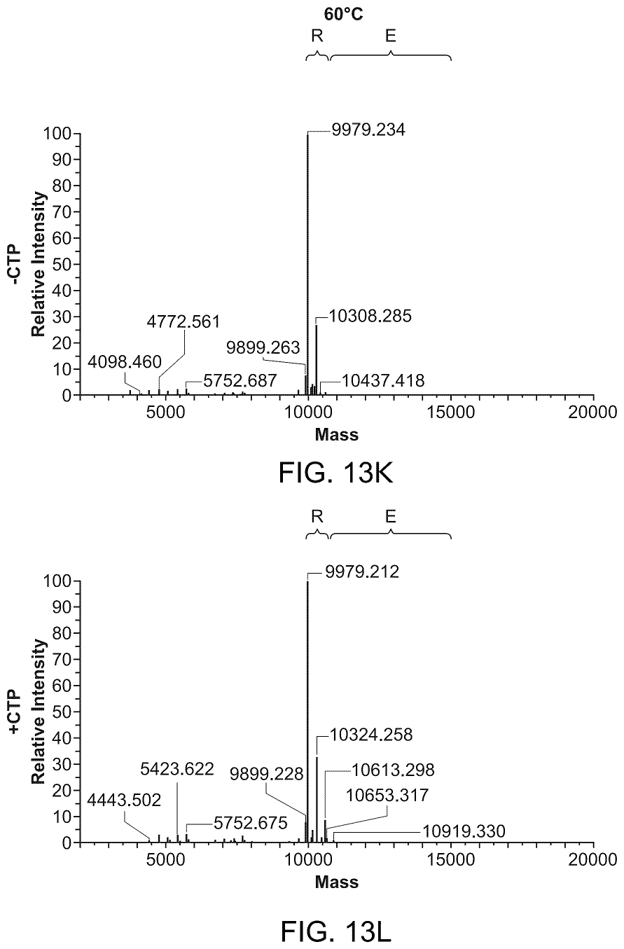

A DNA template of 30-mer was used in IVT reactions according to EXAMPLE 3 in the absence and presence of CTP. Reaction products were purified using Monarch® RNA clean-up kit (NEB, T2040) and subjected to LC/MS intact mass analysis. Nucleic acids in the samples were separated using a Thermo DNAPac™ RP Column on a Vanquish Horizon UHPLC System, followed by mass determination using a Thermo Fisher Scientific Q-Exactive™ Plus mass spectrometer. The raw data were deconvoluted using Promass HR (Novatia, LLC). Example results are shown in FIGS. 6A-6N and 13A-13L.

### Example 5: Capillary Electrophoresis Analysis

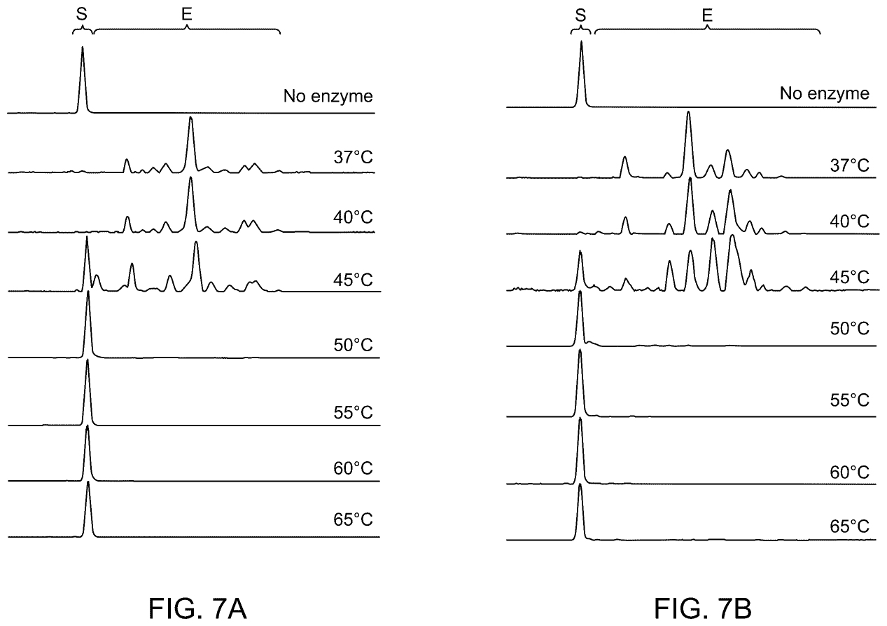

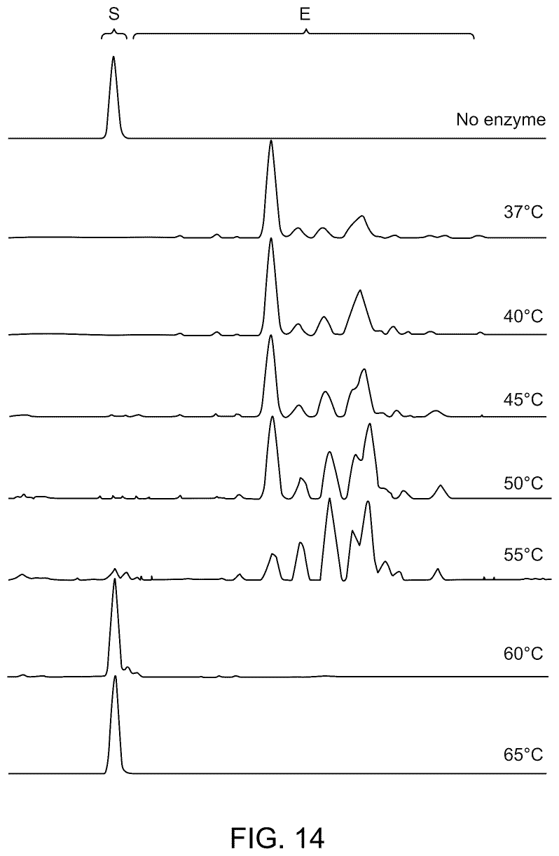

Example capillary electrophoresis reactions were performed by incubating 100 nM 23-nt 5′-FAM oligonucleotide (Integrated DNA Technologies) with RNA polymerases in in vitro transcription buffer at indicated temperatures for 4 hours and quenched in 50 mM Tris-HCl pH 8, 50 mM EDTA and 0.05 Triton X-100. Reaction products were separated by capillary electrophoresis using a 3730x1 Genetic Analyzer (Applied Biosystems) and fluorescent peaks were analyzed using Peak Scanner software version 1.0 (Applied Biosystems). Example results are shown in FIGS. 7A, 7B, and 14.

### Example 6: H6 Mutagenesis and Screening.

A variety of unique multi-mutants [P_{T7}::6×His-H6 RNAP] having an amino-terminal methionine, a 6× histidine tag, and a GS linker (SEQ ID NO: 40) were constructed by incorporating 376 different mutagenic primers into pJAB199 via a previously described extension-ligation strategy (Seyfang and June 2004). The amplified array of multi-mutants were expressed in vitro via NEBExpress®, thermally challenged the lysates at 55° C. for 30 min and monitored activity by production of the fluorescent protein, vGFP (Venus), from pJAB201 [PH6::vGFP] in a subsequent NEBExpress reaction. Linear regression analysis in JMP was used to deconvolute the multi-mutant dataset and calculate the primary (1°) effects of individual substitutions. Results are shown in Table 2. The top 28 hits were further mixed in an additional round of multi-mutant screening. (Table 3) and analysis (Table 4). Using different analytical models that incorporate higher-order interactions (≥2°), optimal substitution combinations were calculated and validated for improved thermostability. Position numbering of Tables 2-4 is according to SEQ ID NO: 40.

TABLE 2
Primary Effect of Substitutions

| Substitution | 1° Effects |
|---|---|
| W768F | 2.668 |
| N547H | 2.238 |
| R525E | 2.069 |
| E726P | 1.303 |
| A493D | 1.275 |
| H857K | 1.071 |
| T241V | 0.654 |
| L143R | 0.578 |
| T338I | 0.554 |
| W807D | 0.505 |
| S468N | 0.442 |
| V411Q | 0.418 |
| M724W | 0.358 |
| Y499L | 0.347 |
| F812I | 0.326 |
| F528S | 0.325 |
| M224I | 0.296 |
| Q336D | 0.295 |
| H117D | 0.269 |
| T387K | 0.171 |
| V411S | 0.152 |
| A490D | 0.116 |
| A428P | 0.065 |
| A489D | −0.024* |
| A463K | −0.050* |
| S765P | −0.068* |
| A115L | −0.079* |
| N292E | −0.333* |
| R44T | −0.544 |
| I485D | −0.609 |
| R381V | −0.684 |
| V67E | −0.687 |
| R400L | −0.729 |
| R400Y | −0.792 |
| V70A | −0.842 |

*omitting an outlier positive result changed the analysis such that the starred residues were >0

TABLE 3
Activity of Variant H6 RNA Polymerases

| | Fold |
|---|---|
| | Increase |
| SUBSTITUTIONS | (x) |
| V411S-A428P-Y499L-N547H-E726P-W768F | 82.145 |
| V411Q-A489D-N547H-M724W-S765P-F812I | 79.634 |
| M224I-T241V-A489D-N547H-F812I-H857K | 72.512 |
| Q336D-V411Q-A490D-F528S-N547H-H857K | 46.810 |
| H117D-M224I-Q336D-A463K-N547H-W807D | 18.962 |
| A115L-T338I-Y499L-R525E-W768F-F812I | 17.909 |
| T387K-V411Q-A463K-A489D-R525E-N547H | 9.816 |
| H117D-T338I-Y499L-N547H-M724W-W807D | 9.150 |
| N292E-T338I-S468N-E726P-W768F-H857K | 8.068 |
| H117D-T241V-V411Q-A428P-F528S-N547H | 5.267 |
| T241V-S468N-A493D-E726P-W768F-W807D | 3.823 |
| N292E-T338I-T387K-S468N-Y499L-R525E | 3.233 |
| M224I-N292E-V411Q-A428P-A490D-W768F | 2.826 |
| T241V-N292E-A463K-W768F-W807D-H857K | 2.471 |
| H117D-M224I-N292E-S468N-A493D-N547H | 1.952 |
| T241V-T387K-Y499L-M724W-S765P-F812I | 1.428 |
| L143R-S468N-A493D-F528S-M724W-F812I | 1.184 |

TABLE 4
Primary Effect of Substitutions

| Substitution | 1° Effects |
|---|---|
| H117D | 0.37 |
| L143R | 1.14 |
| M224I | 0.42* |
| T241V | 1.13 |
| T338I | 0.86 |
| V411Q | 0.69 |
| V411S | 0.45 |
| S468N | 0.44* |
| A490D | 0.71 |
| A493D | 1.55 |
| R525E | 2.86 |
| N547H | 2.14* |
| E726P | 2.20 |
| W768F | 3.40* |
| W807D | 1.31* |
| F812I | 0.77* |
| H857K | 1.56* |

*comprise 7step, computed to be the best overall mutant

What is claimed is:

1. A variant H6 RNA polymerase having (a) RNA polymerase activity, and (b) an amino acid sequence having (i) at least 98% identity to any of SEQ ID NOS: 1-20, and (ii) at least one substitution relative to SEQ ID NO: 21.

2. A variant H6 RNA polymerase having (a) RNA polymerase activity, and (b) an amino acid sequence having (i) at least 95% identity to any of SEQ ID NOS: 1-20, and (ii) at least one substitution relative to SEQ ID NO: 21 at a position selected from positions corresponding to positions 106, 108, 134, 215, 232, 283, 327, 329, 378, 402, 419, 454, 459, 480, 481, 484, 490, 516, 519, 538, 715, 717, 756, 759, 798, 803, and 848 of SEQ ID NO: 21.

3. A variant H6 RNA polymerase having (a) RNA polymerase activity, and (b) an amino acid sequence having (i) at least 95% identity to any of SEQ ID NOS: 1-20, and (ii) at least one substitution relative to SEQ ID NO: 21 at a position selected from positions corresponding to positions 215, 459, 538, 759, 798, 803, and 848 of SEQ ID NO: 21.

4. A variant H6 RNA polymerase according to claim 1 further comprising a second substitution at a position (A) other than the position of the first substitution and (B) corresponding to positions 215, 459, 538, 759, 798, 803, and 848 of SEQ ID NO: 21.

5. A variant H6 RNA polymerase according to claim 4 further comprising a third substitution at a position (A) other than the position of the first and second substitutions and (B) corresponding to positions 215, 459, 538, 759, 798, 803, and 848 of SEQ ID NO: 21.

6. A variant H6 RNA polymerase having (a) RNA polymerase activity, and (b) an amino acid sequence having (i) at least 95% identity to any of SEQ ID NOS: 1-20, and (ii) substitutions relative to SEQ ID NO: 21 at positions corresponding to positions 215, 459, 538, 759, 798, 803, and 848 of SEQ ID NO: 21.

7. A fusion comprising, in an N-terminal to C-terminal direction, (I) a purification tag or a sorting signal peptide, and (II) an H6 RNA polymerase according to claim 1 operably linked to (I); or comprising, in an N-terminal to C-terminal direction, (III) an H6 RNA polymerase according to claim 1, and (IV) a purification tag or a sorting signal peptide operably linked to (III).

8. A composition comprising an H6 RNA polymerase according to claim 1.

9. A composition according to claim 8 further comprising a template encoding an RNA of interest.

10. A composition according to claim 9, wherein the template further comprises an H6 RNA polymerase promoter having the sequence of SEQ ID NO: 23.

11. A composition according to claim 8 further comprising at least one of a buffering agent and a polyamine.

12. A composition according to claim 11, wherein the composition comprises both the buffering agent and the polyamine.

13. A composition according to claim 11, wherein the buffering agent comprises HEPES, MES, MOPS, TAPS, tricine, Tris, ACES, ADA, BES, Bicine, CAPS, carbonic acid/bicarbonic acid, CHES, citric acid, DIPSO, EPPS, histidine, MOPSO, phosphoric acid, PIPES, POPSO, TAPS, TAPSO, or triethanolamine.

14. A composition according to claim 11, wherein the polyamine comprises spermidine, spermine, putrescine, polyethylenimine, 1,4,7-triazacyclononane, cyclen, ethylenediamine, or 1,3,5,-triazinane.

15. A composition comprising a fusion according to claim 7.

16. A composition according to claim 15 further comprising a template encoding an RNA of interest.

17. A method of making an RNA of interest, the method comprising:
contacting:
an H6 RNA polymerase according to claim 1;
a template encoding the RNA of interest;
optionally, one or more NTPs;
optionally, one or more modified NTPs; and
optionally, a buffer,
to produce the RNA of interest, wherein the contacting is at a temperature in a range of 45° C.-65° C. and/or for a time in the range of seconds to hours and/or the RNA of interest is a therapeutic RNA.

18. A method according to claim 17, wherein the template further comprises an H6 RNA polymerase promoter having the sequence of SEQ ID NO: 23.

19. A method according to claim 17, wherein the RNA of interest comprises a capped RNA and contacting further comprising contacting the polymerase, the template, the optional components, if present, and a capping enzyme to produce the capped RNA.

20. A method according to claim 17 further comprising contacting the RNA of interest with one or more pharmaceutically acceptable additives.

21. A kit comprising:
an H6 RNA polymerase according to claim 1 in a storage buffer; and
one or more NTPs.

22. A kit according to claim 21 further comprising one or more modified NTPs.

23. A kit according to claim 21 further comprising a capping enzyme.

24. A kit according to claim 21 further comprising a reaction buffer, the reaction buffer comprising a buffering agent and a polyamine.

25. A kit according to claim 24, wherein the buffering agent comprises HEPES, MES, MOPS, TAPS, tricine, Tris, ACES, ADA, BES, Bicine, CAPS, carbonic acid/bicarbonic acid, CHES, citric acid, DIPSO, EPPS, histidine, MOPSO, phosphoric acid, PIPES, POPSO, TAPS, TAPSO, or triethanolamine.

26. A kit according to claim 24, wherein the polyamine comprises spermidine, spermine, putrescine, polyethylenimine, 1,4,7-triazacyclononane, cyclen, ethylenediamine, or 1,3,5,-triazinane.

27. A cell-free H6 RNA polymerase having an amino acid sequence at least 95% identical to SEQ ID NO: 21.
